(19) United States
(12) Patent Application Publication
(10) Pub. No.: US 2019/0169634 A1
(43) Pub. Date: Jun. 6, 2019

(54) GENETIC ERASERS

(71) Applicant: Senti Biosciences, Inc., South San Francisco, CA (US)

(72) Inventors: Timothy Kuan-Ta Lu, Cambridge, MA (US); Remus Wong, San Francisco, CA (US)

(21) Appl. No.: 16/319,786

(22) PCT Filed: Jul. 26, 2017
(86) PCT No.: PCT/US2017/043931

§ 371 Date: Jan. 22, 2019

Related U.S. Application Data
(60) Provisional application No. 62/366,755, filed on Jul. 26, 2016.

(51) Int. Cl. C12N 15/85; C12N 15/62; C12N 5/0783; C12N 5/074; C12N 5/0735

(52) U.S. Cl. CPC C12N 15/85; C12N 15/62; C12N 5/0637; C12N 5/0607; C12Y 301/21; C12N 2830/008; C12N 2800/30; C12N 2800/24; C12N 2310/20; C12N 5/0606

(57) ABSTRACT

Provided herein, in some embodiments, are methods, compositions, systems and kits that enable removal of heterologous nucleic acid from engineered cells.

### RELATED APPLICATION

This application claims the benefit under 35 U.S.C. § 119(e) of U.S. provisional application No. 62/366,755, filed Jul. 26, 2016, which is incorporated by reference herein in its entirety.

### SUMMARY

Provided herein, in some embodiments, are methods, compositions, systems and kits for highly effective removal of heterologous nucleic acid (e.g., DNA) from engineered cells (e.g., used in cell therapies). This technology is broadly transformative for establishing, engineering, manufacturing, and deploying next-generation human cell therapies.

Engineered (synthetic/artificial) genetic (gene) circuits generally are useful, for example, for ex vivo differentiation and manufacturing of cell therapies. Cells harboring engineered genetic circuits (heterologous genetic circuits) that enable the dynamic control of cell function may be used in patients as therapeutics for treating/preventing many different conditions. Once the desired cell types are created and deployed into patients, however, the engineered circuits may not be necessary for the therapeutic application and may even have adverse effects. The ability to effectively remove these genetic circuits (e.g., heterologous DNA cassettes) after they are deployed into patients is useful for patient safety. For example, the differentiation of stem cells into pancreatic beta-islet cells can be enhanced through genetic circuits³, but once the beta-islet cells are introduced into patients, removal of the genetic circuits would reduce regulatory and safety concerns. The multilayer platform as provided herein, which in some embodiments combines several genetic excision and/or degradation technologies, is engineered for efficient removal (“erasing”) of heterologous nucleic acid (e.g., DNA). Thus, the engineered genetic constructs of the present disclosure may be referred to as “genetic erasers.”

Aspects of the present disclosure provide engineered genetic constructs (genetic erasers) comprising an expression cassette (a cassette) that comprises (a) a nucleotide sequence encoding a first product of interest (e.g., a recombinase or nuclease), and (b) a nucleotide sequence encoding a second product of interest (e.g., a therapeutic molecule) and a counterselectable marker (e.g., a prodrug), wherein expression or activity of the first product of interest is activatable, and wherein the first product of interest modulates excision or degradation of the cassette. In some embodiments, component (a) is upstream from component (b).

In some embodiments, the engineered genetic constructs (genetic erasers) comprise an expression cassette (a cassette) that comprises (a) a nucleotide sequence encoding a recombinase, (b) a nucleotide sequence encoding a nuclease, and (b) a nucleotide sequence encoding a product of interest (e.g., a therapeutic molecule) and a counterselectable marker (e.g., a prodrug), wherein the recombinase and nuclease are activatable and modulates excision and/or degradation of the cassette.

In some embodiments, the engineered genetic constructs (genetic erasers) comprise a cassette that comprises (a) an inducible promoter operably linked to a nucleotide sequence encoding a recombinase, and (b) a promoter operably linked to a nucleotide sequence encoding a product of interest and a counterselectable marker, wherein the cassette is flanked by cognate recombinase recognition sites.

In some embodiments, the engineered genetic constructs (genetic erasers) comprise a cassette that comprises (a) an inducible promoter operably linked to a nucleotide sequence encoding a nuclease, and (b) a promoter operably linked to a nucleotide sequence encoding a product of interest and a counterselectable marker, wherein the cassette is flanked by nuclease recognition sites.

In some embodiments, the engineered genetic constructs (genetic erasers) comprise a cassette that comprises (a) an inducible promoter operably linked to a nucleotide sequence encoding a recombinase, (b) an inducible promoter operably linked to a nucleotide sequence encoding a nuclease, and (c) a promoter operably linked to a nucleotide sequence encoding a product of interest and a counterselectable marker, wherein the cassette is flanked by cognate recombinase recognition sites and includes cognate nuclease recognition sites.

Some aspects of the present disclosure provide methods comprising introducing into a cell of any one of the engineered genetic constructs as described herein, wherein the product of interest aids in differentiation, expansion or phenotypic maintenance of the cell.

Other aspects of the present disclosure provide methods comprising introducing into a cell of any one of the engineered genetic constructs as described herein, wherein the product of interest is a therapeutic molecule and/or prophylactic molecule.

The present disclosure also provides vectors, cells, compositions and kits comprising any of the engineered genetic constructs as described herein.

### BRIEF DESCRIPTION OF THE DRAWINGS

FIG. 1 shows an example of how multiple redundant and orthogonal recombinase proteins are utilized to excise heterologous DNA constructs from target cells. These recombinases are controlled through inducible transcription, reconstitution of split protein activity, or translocation of protein to the nuclease.

FIGS. 2A-2B show an example of how nucleases are used to target DNA for (FIG. 2A) destruction and/or (FIG. 2B) trigger recombination to excise large DNA fragments located in between nuclease target sites (NTS). These nucleases can include meganucleases, CRISPR-Cas nucleases, zinc-finger nucleases, and TALE nucleases. Double-stranded breaks induced by nucleases can result in recombination that inactivates targeted protein-coding sequences or catalyzes large deletions, which can be further enhanced through the presence of donor DNA.

FIG. 3 shows an example of counterselectable markers (CSMs) encoded in heterologous DNA constructs so that cells that do not undergo efficient DNA excision with the recombinase-based or nuclease-based approaches are killed upon addition of a prodrug that is converted into a toxic drug. In addition, inducible kill switches are encoded that trigger cell death upon addition of a small-molecule inducer that expresses toxins or dimerizes split toxins. These CSMs will be utilized in combination with the genetic erasers to enhance the efficiency of heterologous DNA deletion.

FIG. 4 shows an example of a recombinase-based excision construct for stable integration. This modular construct enables easy exchange of genetic parts by simply digesting the vector with difference combinations of restriction enzymes.

FIG. 5 shows an example of a system to assay for recombinase excision efficiency. The reporter vector encodes recombination sequences flanking a counter-selection marker (e.g., HSV thymidine kinase). DNA assembly may be performed with multi-step approaches including, for example, restriction enzyme cloning, Gibson assembly and Golden Gate assembly. The recombinase vector encodes a recombinase (e.g., Bxb1).

FIG. 6 shows data from a transient 293FT cell transfection assay to test recombinase activity of various tyrosine recombinases. 293FT cells were transiently transfected with an equal ratio of reporter plasmid, recombinase plasmid, and a transfection marker plasmid (e.g., a plasmid encoding BFP). The cells were assayed for GFP fluorescence 24 hours post transfection and gated for BFP expression. The top graph represents % GFP+ cells, and the bottom graph represents the median of the GFP mean fluorescence intensity of the GFP+ cells. Each group of bars shows, left to right, data for: reporter, recombinase, and reporter+recombinase.

FIG. 7 shows data from a transient 293FT cell transfection assay to test recombinase activity of various tyrosine recombinases. 293FT cells were transiently transfected with an equal ratio of reporter plasmid, recombinase plasmid, and a transfection marker plasmid (e.g., a plasmid encoding BFP). The cells were assayed for GFP fluorescence 24 hours post transfection and gated for BFP expression. The histograms represent transfected cell populations with and without recombinase.

FIG. 8 shows an example of a system used to assay for recombinase excision efficiency. A BpiI(x2)-HSVtk-SV40 pA-EGFP-Esp3I(x2) cassette was inserted into the pcDNA3.1(+) mammalian expression vector (Life Tech). Recombination sequences were inserted into the BpiI and Esp3I sites via Golden Gate digestion/ligation.

FIG. 9 shows data from a transient 293FT cell transfection assay to test recombinase activity of various serine integrases. 293FT cells were transiently transfected with an equal ratio of reporter plasmid, recombinase plasmid, and a transfection marker plasmid (e.g., a plasmid encoding BFP). The cells were assayed for GFP fluorescence 24 hours post transfection and gated for BFP expression. The top graph represents % GFP+ cells, and the bottom graph represents the median of the GFP mean fluorescence intensity of the GFP+ cells. Each group of bars shows, left to right, data for: reporter, recombinase, and reporter+recombinase.

FIG. 10 shows data from a transient 293FT cell transfection assay to test expression of various serine integrases. 293FT cells were transiently transfected with an equal ratio of reporter plasmid, recombinase plasmid, and a transfection marker plasmid (e.g., a plasmid encoding BFP). The cells were assayed for GFP fluorescence 24 hours post transfection and gated for BFP expression. The histograms represent transfected cell populations with and without recombinase.

FIG. 11 shows an example of a recombinase-based excision construct for site-specific integration. An entry vector encoding a recombinase-based excision construct is integrated into a pre-engineered 293FT landing pad cell line that expresses YFP and hygromycin. Successful integration drives expression of puromycin.

FIG. 12 shows successful integration of a GFP reporter into a 293FT landing pad cell line. Upon integration, cells express GFP and simultaneously lose expression of YFP. Selection pressure with puromycin removes unintegrated cells.

FIG. 13 shows an example of a method for excising genomically-integrated constructs. An entry vector encoding a recombinase-based excision construct is integrated into a pre-engineered 293FT landing pad cell line. Integrated cell lines are transiently transfected with a recombinase-expressing plasmid and a reporter plasmid (e.g., expressing BFP) and assayed for GFP expression over time. A counter-selection marker (CSM) is used to kill off cells that retain the integrated construct.

FIG. 14 shows example data obtained using the experimental method described in FIG. 13. Cell lines expressing 3 different recombinase-based excision constructs were transiently transfected with the cognate recombinase, and GFP expression was assayed over time. The % of GFP+ cells were plotted for cell populations that were either ungated or gated for BFP expression. Each group of bars shows, left to right, data for: day 2, day 4, and day 7.

FIG. 15 shows example data obtained using the experimental method described in FIG. 13. Following transient transfection of the B3 recombinase in FIG. 14, a prodrug was applied to kill off cells that retained the pENTR_B3RT excision construct. The counter selection marker (CSM) converts the prodrug into a toxic drug. In this case, the CSM was HSVtk and the prodrug was ganciclovir (GCV). The cells were treated with 0.5, 1, 2, and 5 μM GCV for 7 days, and GFP expression was assayed over time. The histograms represent the % of GFP- and % of GFP+ cells.

FIG. 16 shows example data obtained using the experimental method described in FIG. 13. Following transient transfection of the Flp recombinase in FIG. 14, a prodrug was applied to kill off cells that retained the pENTR_FRT excision construct. The counter selection marker (CSM) converts the prodrug into a toxic drug. In this case, the CSM was HSVtk and the prodrug was ganciclovir (GCV). The cells were treated with 0.5, 1, 2, and 5 μM GCV for 7 days, and GFP expression was assayed over time. The histograms represent the % of GFP- and % of GFP+ cells.

FIG. 17 shows an example of a system in which guide RNAs (gRNAs) cleave and remove a genomically-integrated circuit. In this figure, the gRNAs target the 5′-UTR and 3′-UTR if a YFP reporter that has been stably integrated into cells.

FIG. 18 shows data from a transient transfection assay to test the removal of a genomically-integrated circuit using CRISPR/Cas9. Vectors encoding a single gRNA and Cas9 were co-transfected along with a reporter plasmid (e.g., expressing BFP) into a cell line that expresses YFP. In some cases, two vectors encoding different gRNAs were transfected along with a reporter plasmid. The cell populations were gated for BFP expression, and the % of YFP+ cells were plotted over time. Different combinations of gRNAs may be used to remove YFP. The histograms represent the % of YFP− and % of YFP+ cells. Each group of bars shows, left to right, data for: day 2, day 5, and day 7.

FIG. 19 shows example data obtained using the experimental method described in FIG. 13. Cell lines expressing the pENTR_B3RT_FRT recombinase-based excision construct were sequentially transfected with B3 or Flp according to the indicated timeline. GFP expression was assayed over time, and the histograms represent the % of GFP+ cells on day 15. The pENTR_B3RT_FRT recombinase-based excision construct encodes the construct B3RT_FRT_iCasp9_SV40 pA_FRT_B3RT_EGFP_BGHpA, in which B3RT and FRT are recombination sequences for B3 and Flp, respectively; and iCasp9 is a counter selection marker (CSM).

FIG. 20 shows example data obtained using the experimental method described in FIG. 13. Cell lines expressing the pENTR_B3RT_FRT recombinase-based excision construct were sequentially transfected with B3 or Flp according to the indicated timeline. GFP expression was assayed over time, and the % of GFP+ cells on day 15 were plotted. Bars show, left to right, data for: no rec; B3; B3, Flp; Flp; and Flp, B3.

### DETAILED DESCRIPTION

Described herein is a powerful technology that enables next-generation cell therapies, in part, by enabling highly efficient removal of heterologous nucleic acid from engineered cells. The engineered genetic constructs herein may be referred to as “genetic erasers.” The genetic eraser technologies described herein are useful for at least two major applications. First, they are useful for creating genetic circuits that aid in the differentiation, expansion, or phenotypic maintenance of cells ex vivo, which can then be removed prior to being introduced into patients. Second, they are used as safety switches that can be triggered after cell therapies are delivered in vivo by removing heterologous DNA from engineered cells so that the cell therapies lose their function.

Thus, in some aspects, these genetic erasers enable the implementation of genetic circuits that enhance the differentiation, expansion, or persistence of specific phenotypes ex vivo and subsequent removal the genetic circuits before introduction into the body. Existing strategies for regulating cell phenotypes include the use of small molecules, growth factors, RNA constructs, or DNA circuits. Small molecules can be useful for enhancing differentiation but discovering such chemicals can be laborious and difficult given the complexities of endogenous cell regulation. Growth factors have been used successfully to program cells ex vivo but can be expensive to discover, scale, and apply given the many potential combinations. RNA constructs encoding transcription factors and other intracellular regulators of cell function have been utilized for cell programming, but have not been used to encode complex dynamics into cell programs that are necessary for improved efficiency due to the lack of programmable RNA regulators (albeit, this is beginning to change with recent advancements (25)). DNA circuits have been used to program complex transcriptional programs, such as the differentiation of progenitor cells derived from iPS cells into beta-islet-like cells (3), as discussed above. These circuits can be used to express intracellular regulators, such as transcription factors and microRNAs, as well as secrete paracrine factors such as growth factors and cytokines, and thus have the potential to increase the scale and reduce the cost of ex vivo cellular programming. The removal of such circuits prior to introduction into the patient would reduce concerns over safety and regulatory burden.

The genetic erasers of the present disclosure, in some embodiments, integrate multiple mechanisms to achieve significant levels of genetic erasure (deletion/degradation), which can restore cells to a baseline state without heterologous activities and can restore the genome to a baseline state with almost no trace of foreign DNA (recombinases may leave small DNA scars with minimal effects). Using multiple redundant layers of genetic erasers enables significantly improved efficiencies of genetic deletion and is important for enabling clinical applications (achieving clinical-grade activity) of this technology.

For the various approaches described herein, efficiencies of removing heterologous DNA or killing cells that contain heterologous DNA are measured. In the former, fluorescence or qPCR-based assays are used. In the latter, cell survival is monitored using live-dead assays. This is prototyped in cell lines and extended into therapeutically relevant cell types (e.g., mesenchymal stem cells, T cells, NK cells). Such technologies are useful for programming efficient cell differentiation and then removing these circuits prior to therapy. Such technologies also are useful as kill switches or OFF switches for in vivo applications of genetically engineered cell therapies.

A key technical challenge for genetic erasers is to minimize the number of cells that escape from the genetic erasing process. Doses of CAR T cells in trials range from 2*10⁵-2*10⁷ CD19 CART cells/kg, whereas mesenchymal stem cells (MSCs) being used in the clinic are in the range of 1-3*10⁶ MSCs/kg (27). Thus, maximal doses of cell therapies being used in the clinic appear to be less than 10⁹ cells on the high end. To ensure that less than 1 cell per dose contains heterologous genetic material, this means that, in some embodiments, genetic erasers that can achieve efficiencies of greater than 10⁹ are preferred. To achieve this stringent efficiency, multiple genetic erasure mechanisms can be layered together, as provided herein. Combinations of genetic eraser components lead to enhanced efficiencies. For example, if greater than 97% excision efficiency is achieved with four different recombinases and greater than 97% killing efficiency is achieved with two counterselectable markers, then only one cell in ˜7.3¹⁰ cells would remain after application of genetic eraser technology. In some embodiments, conditionally replicating plasmids are used instead of encoding genetic circuits into the genome to improve the efficiency of removing heterologous DNA or RNA circuits with new dynamic regulators (25).

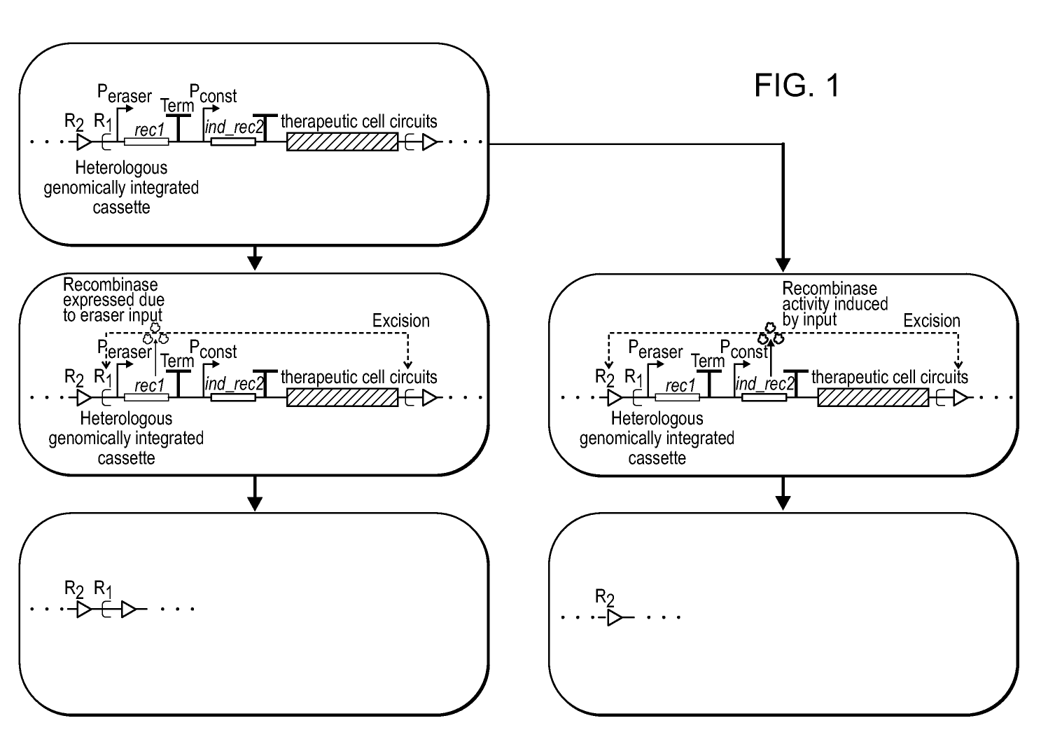

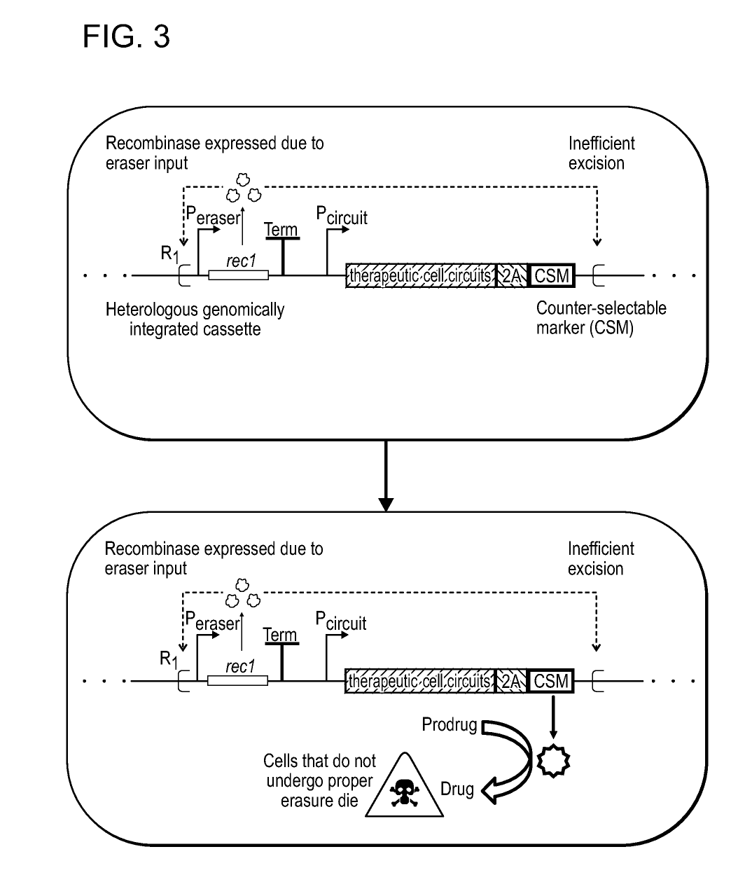

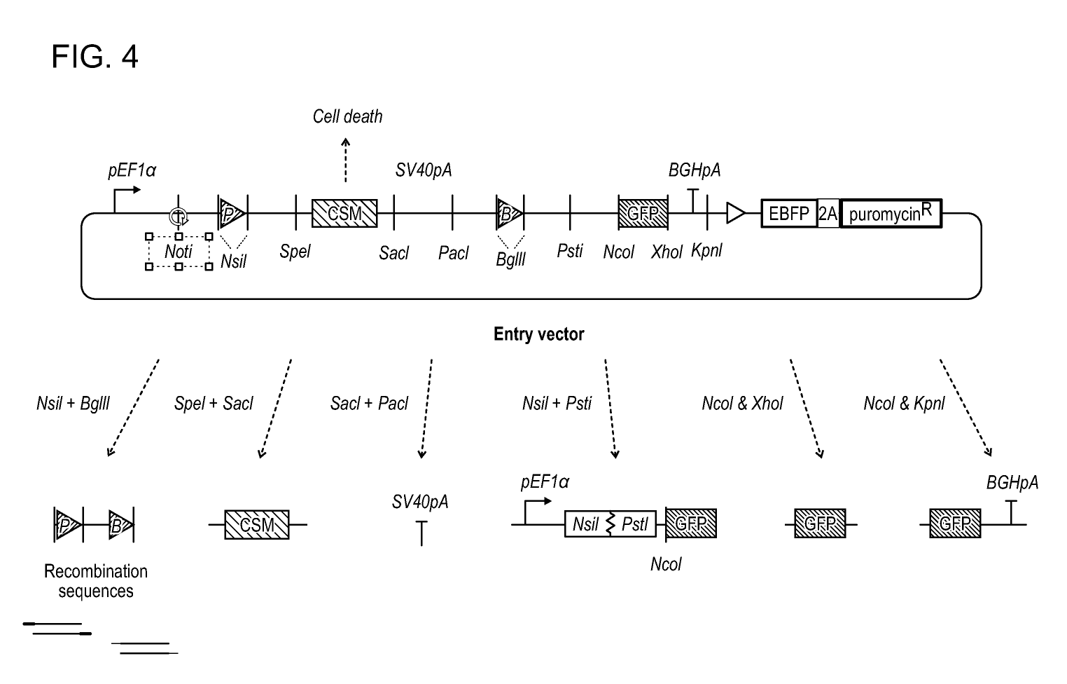

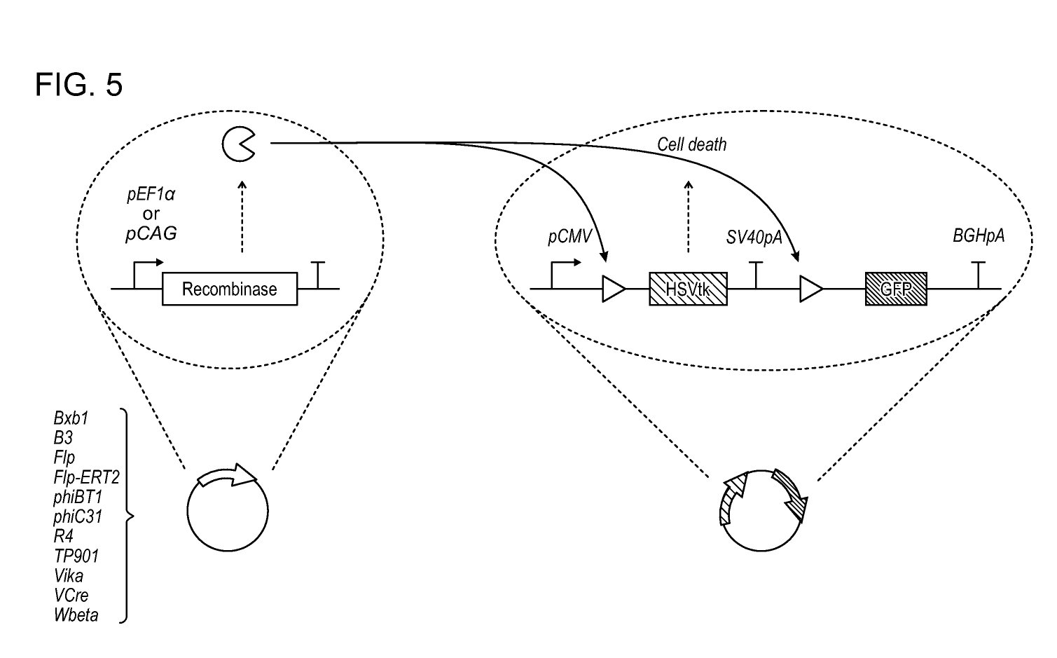

Thus, provided herein are engineered genetic constructs comprising a cassette that comprises (a) an inducible promoter operably linked to a nucleotide sequence encoding a recombinase, and (b) a promoter operably linked to a nucleotide sequence encoding a product of interest and a counterselectable marker, wherein the cassette is flanked by cognate recombinase recognition sites. The recombinase functions to self-excise the cassette, and the counterselectable marker functions to kill off any cells in which the cassette remains following an excision event. In some embodiments, the inducible promoter operably linked to a nucleotide sequence encoding a recombinase is upstream from (5′ from) the promoter operably linked to a nucleotide sequence encoding a product of interest. See, e.g., FIGS. 1 and 3.

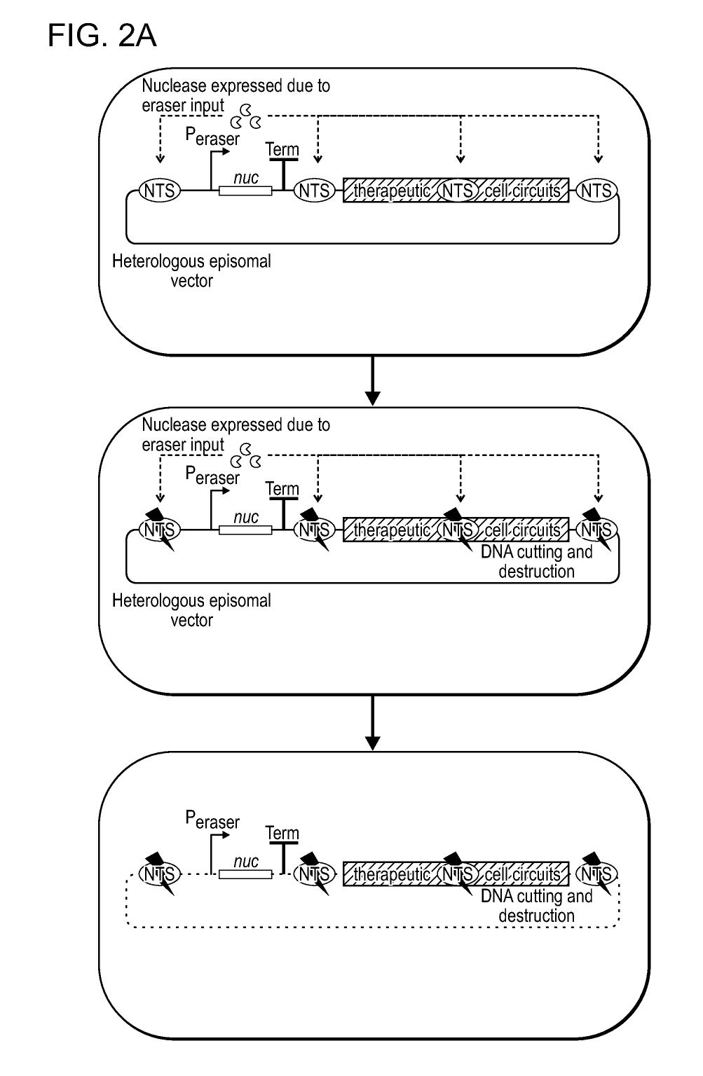

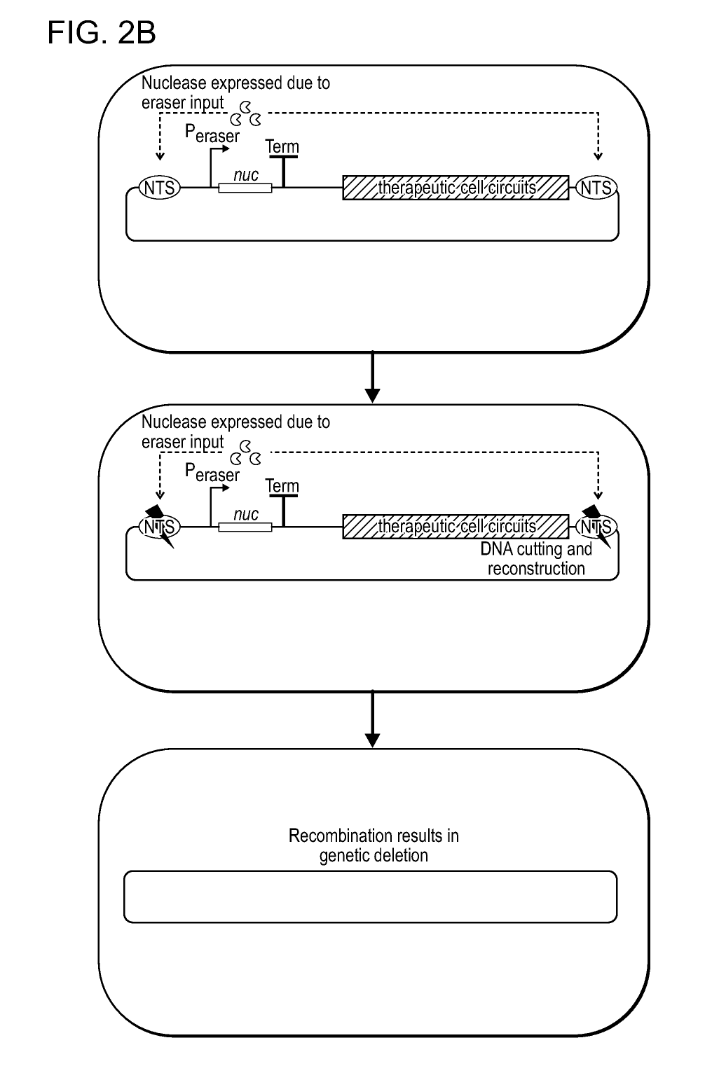

Also provided herein are engineered genetic constructs comprising a cassette that comprises (a) an inducible promoter operably linked to a nucleotide sequence encoding a nuclease, and (b) a promoter operably linked to a nucleotide sequence encoding a product of interest and a counterselectable marker, wherein the cassette comprises cognate nuclease recognition sites. The nuclease functions to cut up/degrade the cassette, and the counterselectable marker, and the counterselectable marker functions to kill off any cells in which the cassette remains following a degradation event. In some embodiments, the inducible promoter operably linked to a nucleotide sequence encoding a nuclease is upstream from (5′ from) the promoter operably linked to a nucleotide sequence encoding a product of interest. See, e.g., FIGS. 2 and 3.

As discussed above, the technology of present disclosure includes a multilayered platform. Thus, a single genetic construct encoding a product of interest may encode multiple (e.g., at least 2, 3, 4, or 5) different recombinases, nucleases and/or counterselectable markers. Likewise, the same genetic construct may include multiple pairs of recombinase recognition sites and/or multiple nuclease recognitions sites to enable highly efficient excision and/or degradation of the nucleic acid encoding the different recombinases and/or nucleases and the product of interest.

In some embodiments, a single construct encodes a recombinase, a nuclease and a counterselectable marker. In some embodiments, a single construct encodes at least one (e.g., 1, 2, 3, 4 or 5) recombinase, at least one (e.g., 1, 2, 3, 4 or 5) nuclease and at least one (e.g., 1, 2, 3, 4 or 5) counterselectable marker. In some embodiments, a single construct encodes at least one recombinase and at least one nuclease. In some embodiments, a single construct encodes at least one recombinase and at least one counterselectable marker. In some embodiments, a single construct encodes at least one nuclease and at least one counterselectable marker. In some embodiments, a single construct encodes at least two recombinases and at least one nuclease. In some embodiments, a single construct encodes at least two recombinases and at least one counterselectable marker. In some embodiments, a single construct encodes at least two nucleases and at least one recombinase. In some embodiments, a single construct encodes at least two nucleases and at least one counterselectable marker. In some embodiments, a single construct encodes at least two recombinases, at least two nucleases and at least one counterselectable marker. In some embodiments, a single construct encodes at least two recombinases, at least two nucleases and at least two counterselectable markers.

Thus, in some embodiments, a cassette of a single construct may be flanked by multiple pairs of cognate recombinase recognition site and/or may contain multiple nuclease recognition sites.

Further, in some embodiment, an engineered genetic construct comprising a cassette that comprises at least two inducible promoters, each linked to a nucleotide sequence encoding a different recombinase and/or nuclease, wherein the cassette is flanked by recombinase recognition sites cognate to the different recombinases and/or comprises nuclease recognition sites cognate to the different nucleases. In some embodiments, the inducible promoter is linked to at least two nucleotide sequences, each encoding a different recombinase and/or nuclease, and wherein the cassette is flanked by recombinase recognition sites cognate to the different recombinases and/or comprises nuclease recognition sites cognate to the different nucleases. In some embodiments, the construct comprises at least three inducible promoters, each linked to a nucleotide sequence encoding a different recombinase and/or nuclease, wherein the cassette is flanked by recombinase recognition sites cognate to the different recombinases and/or comprises nuclease recognition sites cognate to the different nucleases. In some embodiments, the inducible promoter is linked to at least three nucleotide sequences, each encoding a different recombinase and/or nuclease, wherein the cassette is flanked by recombinase recognition sites cognate to the different recombinases and/or comprises nuclease recognition sites cognate to the different nucleases.

As discussed above, the different combinations of genetic eraser components lead to enhanced efficiencies in removal of heterologous nucleic acid. Following a series of excision, degradation and/or counterselection reactions (which include exposing the cells to all inducing agents and/or counterselective agents necessary to express each component of the expression cassette), in some embodiments, less than 20% (e.g., 19%, 18%, 17%, 16%, 15%, 14%, 13%, 12%, 11%, 10%, 9%, 8%, 7%, 6%, 5%, 4%, 3%, 2%, 1% or 0%) of the cells that initially received the engineered genetic constructs retain the expression cassettes. That is, greater than 80% (e.g., 81%, 82%, 83%, 84%, 85%, 86%, 87%, 88%, 89%, 90%, 91%, 92%, 93%, 94%, 95%, 96%, 97%, 98%, 99%, or 100%) of the cells no longer contain the expression cassette following a series of excision, degradation and/or counterselection reactions. In some embodiments 1-20%, 1-15%, 1-10%, 1-5%, 5-20%, 5-15%, 5-10%, 10-20% or 15-20% of the cells that initially received the engineered genetic constructs retain the expression cassettes following a series of excision, degradation and/or counterselection reactions. In some embodiments 80-100%, 80-95%, 80-90%, 80-85%, 85-100%, 85-95%, 85-90%, 90-100% or 95-100% of the cells that initially received the engineered genetic constructs no longer contain the expression cassette following a series of excision, degradation and/or counters election reactions.

Thus, in some embodiments, methods of the present disclosure may include introducing into a population of cells an engineered genetic construct as provided herein, culturing the cells to produce the product of interest, culturing the cells in the presence of at least one (one or more) inducer agent to express the recombinase and/or nuclease to excise and/or degrade the cassette from the engineered genetic construct, culturing cells of the population in the presence of a counterselective agent to kill cells that still retain and express the counterselectable marker. An inducer agent is any agent that activates a cognate inducible promoter (the activity of which is activated in the presence of the inducer). A counterselective agent is any agent that kills a cell that expresses/contains the counterselectable marker (which is toxic to the cells, e.g., is converted to a toxic agent, in the presence of the counterselective agent).

### Recombinase-Based Excision

In some embodiments, an engineered genetic construct comprises an expression cassette (a cassette) that comprises (a) an inducible promoter operably linked to a nucleotide sequence encoding a recombinase, and (b) a promoter operably linked to a nucleotide sequence encoding a product of interest and a counterselectable marker, wherein the cassette is flanked by cognate recombinase recognition sites. In some embodiments, component (a) is upstream from component (b). In some embodiments, a terminator sequence is located between component (a) and component (b). See, e.g., FIG. 1.

In some embodiments, component (a) comprises at least two (or at least three) inducible promoters, each linked to a different recombinase, and the cassette is flanked by recombinase recognition sites cognate to the different recombinases. In some embodiments, the nucleotide sequence of component (a) encodes at least two (or at least three) different recombinases, and the cassette is flanked by recombinase recognition sites cognate to the different recombinases. In some embodiments, the nucleotide sequence of component (b) encodes at least two (or at least three) counterselectable markers.

In some embodiments, the recombinase(s) is selected from tyrosine recombinases and tyrosine integrases. For example, the recombinase(s) may be selected from Cre, Dre, Flp, KD, B2, B3, λ, HK022 and HP1 recombinases. In some embodiments, the recombinase(s) is selected from serine recombinases or serine integrases. For example, the recombinase(s) may be selected from γδ, ParA, Tn3, Gin, ΦC31, Bxb1 and R4 recombinases.

A recombinase is a site-specific enzyme that recognizes short DNA sequence(s), typically between about 30 base pairs (bp) and 40 bp, that mediates the recombination between these recombinase recognition sequences, which results in the excision, integration, inversion, or exchange of DNA fragments between the recombinase recognition sequences.

Recombinases can be classified into two distinct families: serine recombinases (e.g., resolvases and invertases) and tyrosine recombinases (e.g., integrases), based on distinct biochemical properties. Serine recombinases and tyrosine recombinases are further divided into bidirectional recombinases and unidirectional recombinases. Examples of bidirectional serine recombinases include, without limitation, β-six, CinH, ParA and γδ; and examples of unidirectional serine recombinases include, without limitation, Bxb1, ϕC31, TP901, TG1, φBT1, R4, φRV1, φFC1, MR11, A118, U153 and gp29. Examples of bidirectional tyrosine recombinases include, without limitation, Cre, FLP, and R; and unidirectional tyrosine recombinases include, without limitation, Lambda, HK101, HK022 and pSAM2. The serine and tyrosine recombinase names stem from the conserved nucleophilic amino acid residue that the recombinase uses to attack the DNA and which becomes covalently linked to the DNA during strand exchange. Recombinases have been used for numerous standard biological applications, including the creation of gene knockouts and the solving of sorting problems.

The outcome of recombination depends, in part, on the location and orientation of two short repeated DNA sequences that are to be recombined, typically less than 30 bp long. Recombinases bind to these repeated sequences, which are specific to each recombinase, and are herein referred to as recombinase recognition sequences or recombinase recognition sites. Thus, as used herein, a recombinase is specific for a recombinase recognition site when the recombinase can mediate inversion or excision between the repeat DNA sequences. As used herein, a recombinase may also be said to recognize its cognate recombinase recognition sites, which flank an intervening genetic element (e.g., promoter, terminator, or output nucleic acid sequence). A nucleic acid or fragment of a nucleic acid is said to be flanked by recombinase recognition sites when the element is located between and immediately adjacent to two repeated DNA sequences. In some embodiments, the recombinase recognition sites do not overlap each other. However, in other embodiments, recombinase recognition sites do overlap each other, which permits greatly increased combinatorial complexity.

Inversion recombination happens between two short, inverted, repeated DNA sequences. A DNA loop formation, assisted by DNA bending proteins, brings the two repeat sequences together, at which point DNA cleavage and ligation occur. This reaction is ATP independent and requires supercoiled DNA. The end result of such an inversion recombination event is that the stretch of DNA between the repeated site inverts (i.e., the stretch of DNA reverses orientation) such that what was the coding strand is now the non-coding strand and vice versa. In such reactions, the DNA is conserved with no net gain or no loss of DNA.

Conversely, integration (excision) recombination occurs between two short, repeated DNA sequences that are oriented in the same direction. In this case, the intervening DNA is excised/removed.

Recombinases can also be classified as irreversible or reversible. An irreversible recombinase is a recombinase that can catalyze recombination between two complementary recombination sites, but cannot catalyze recombination between the hybrid sites that are formed by this recombination without the assistance of an additional factor. Thus, an irreversible recognition site refers to a recombinase recognition site that can serve as the first of two DNA recognition sequences for an irreversible recombinase and that is modified to a hybrid recognition site following recombination at that site. A complementary irreversible recognition site refers to a recombinase recognition site that can serve as the second of two DNA recognition sequences for an irreversible recombinase and that is modified to a hybrid recombination site following homologous recombination at that site. For example, attB and attP, are the irreversible recombination sites for Bxb1 and phiC31 recombinases—attB is the complementary irreversible recombination site of attP, and vice versa. AttB/attP sites can be mutated to create orthogonal B/P pairs that only interact with each other but not the other mutants. This allows a single recombinase to control the excision or integration or inversion of multiple orthogonal B/P pairs.

The phiC31 (φC31) integrase, for example, catalyzes only the attB×attP reaction in the absence of an additional factor not found in eukaryotic cells. The recombinase cannot mediate recombination between the attL and attR hybrid recombination sites that are formed upon recombination between attB and attP. Because recombinases such as the phiC31 integrase cannot alone catalyze the reverse reaction, the phiC31 attB×attP recombination is stable.

Irreversible recombinases, and nucleic acids that encode the irreversible recombinases, are described in the art and can be obtained using routine methods. Examples of irreversible recombinases include, without limitation, phiC31 (φC31) recombinase (SEQ ID NO:11), coliphage P4 recombinase, coliphage lambda integrase, Listeria A118 phage recombinase, and actinophage R4 Sre recombinase, HK101, HK022, pSAM2, Bxb1, TP901, TG1, φBT1, φRV1, φFC1, MR11, U153 and gp29.

Conversely, a reversible recombinase is a recombinase that can catalyze recombination between two complementary recombinase recognition sites and, without the assistance of an additional factor, can catalyze recombination between the sites that are formed by the initial recombination event, thereby reversing it. The product-sites generated by recombination are themselves substrates for subsequent recombination. Examples of reversible recombinase systems include, without limitation, the Cre-lox and the Flp-frt systems, R, β-six, CinH, ParA and γ6.

In some embodiments, an engineered genetic construct encodes a recombinase selected from tyrosine recombinases or tyrosine integrases. In some embodiments, the recombinase is selected from Cre, Dre, Flp, KD, B2, B3, λ, HK022 and HP1 recombinases. In some embodiments, an engineered genetic construct encodes a recombinase selected from serine recombinases or serine integrases. In some embodiments, the recombinase is selected from γδ, ParA, Tn3, Gin, ΦC31, Bxb1 and R4 recombinases.

The recombinases provided herein are not meant to be exclusive examples of recombinases that can be used in embodiments of the present disclosure. The complexity of genetic erasers of the present disclosure can be expanded by mining databases for new orthogonal recombinases or designing synthetic recombinases with defined DNA specificities. Other examples of recombinases that are useful are known to those of skill in the art, and any new recombinase that is discovered or generated is expected to be able to be used in the different embodiments of the present disclosure.

Further, control of recombinase expression and/or activation can be achieved many difference ways, using, for example, the ABA system (Liang, F. S., et al. Sci. Signal., 2011, 4, rs2 LP-rs2), the GIB system (Miyamota, T. et al. Nat Chem Biol, 2012, 8, 465-470), the FKBP-FRB based dimerization system (Komatsu, T. et al. Nat Meth, 2010, 7, 206-208), the tamoxifen system (Matsuda, T. et al. Proc. Natl. Acad. Sci., 2007, 104, 1027-1032), the DD(FKBP) system (Banaszynski, L. A. et al. Cell, 2006, 126, 995-1004), the DD(DHFR) system (Iwamoto, M. et al. Chem. Biol., 2010, 17, 981-988), the SMAsh system (Chung, H. K. et al. Nature Chemical Biology, 11: 713-720, 2015), or through blue light induced dimerization (Guntas, G. et al. Proc. Natl. Acad. Sci., 2015, 112, 112-117, each of which is incorporated herein by reference).

In some embodiments, an engineered genetic construct comprising a cassette that comprises (a) a nucleotide sequence encoding at least one ligand-dependent chimeric recombinase, and (b) a nucleotide sequence encoding a product of interest and a counterselectable molecule, wherein the cassette is flanked by cognate recombinase recognition sites.

In some embodiments, an engineered genetic construct comprising a cassette that comprises (a) a nucleotide sequence encoding a first fragment of a recombinase, (b) a nucleotide sequence encoding a second fragment of a recombinase, wherein the first fragment and the second fragment when combined (dimerize) form a full-length functional recombinase (see, e.g., Miyamoto, T. et al. Nature Chemical Biology 8:465-470, 2012, incorporated herein by reference), and (c) a nucleotide sequence encoding a product of interest and a counterselectable marker, wherein the cassette is flanked by cognate recombinase recognition sites.

### Nuclease-Based Excision/Degradation

In some embodiments, an engineered genetic construct comprises an expression cassette (a cassette) that comprises (a) an inducible promoter operably linked to a nucleotide sequence encoding a nuclease, and (b) a promoter operably linked to a nucleotide sequence encoding a product of interest and a counterselectable marker, wherein the cassette comprises cognate nuclease recognition sites. In some embodiments, nuclease recognition sites flank the cassette. In some embodiments, component (a) is upstream from component (b). In some embodiments, a terminator sequence is located between component (a) and component (b). See, e.g., FIG. 2.

In some embodiments, component (a) comprises at least two inducible promoters, each linked to a different nuclease, and the cassette comprises nuclease recognition sites cognate to the different nucleases. In some embodiments, the nucleotide sequence of component (a) encodes at least two different nucleases, and the cassette comprises nuclease recognition sites cognate to the different nucleases. In some embodiments, the nucleotide sequence of component (b) encodes at least two counterselectable markers.

In some embodiments, the nuclease(s) is selected from meganucleases and RNA-guided nucleases. For example, the nuclease(s) may be a meganuclease selected from intron endonucleases and intein endonucleases. In some embodiments, the nuclease(s) is a RNA-guided nuclease selected from Cas9 nucleases and Cpf1 nucleases. Thus, the cassette may further comprise nucleotide sequences encoding guide RNAs (gRNAs) complementary to the nuclease recognitions sites.

A nuclease is an enzyme that cleaves the phosphodiester bonds between monomers of nucleic acids. Many nucleases, such as restriction endonuclease, cleaves DNA at specific sites along the molecule. These sites at which a nuclease cleaves are referred to as nuclease recognition sites. There are many different types of nucleases that may be used in accordance with the present disclosure, including restriction nucleases, such as meganucleases, RNA-guided nucleases, zinc-finger nucleases, and transcription activator-like effector nucleases.

Meganucleases (e.g., I-SceI, I-CreI, I-Dmol, E-Drel and DmoCre) are endodeoxyribonucleases that have a long recognition site (e.g., double-stranded DNA sequences of 12 to 40 base pairs). Thus, this site generally occurs only once in any given genome. There are hundreds of meganucleases known in the art. Meganucleases are mainly represented by two main enzyme families collectively known as homing endonucleases: intron endonucleases and intein endonucleases. In some embodiments, the meganuclease is a LAGLIDADG family endonuclease. The name of this family corresponds to an amino acid sequence (or motif) that is found, generally conserved, in all the proteins of this family.

RNA-guided nucleases are endonucleases that are selectively guided to their target sites by associating with a guide RNA (gRNA) strand that includes a sequence complementary to the target site. In some embodiments, the RNA-guided nuclease is a member of the clustered, regularly interspaced, short palindromic repeats (CRISPR)-CRISPR-associated (Cas) system. In some embodiments, the RNA-guided nuclease is a member of the Type II CRISPR-Cas system. CRISPR-Cas systems are used by various bacteria and archaea to mediate defense against viruses and other foreign nucleic acid. Short segments of foreign DNA, called spacers, are incorporated into the genome between CRISPR repeats and serve as a ‘memory’ of past exposures. CRISPR spacers are then used to recognize and silence exogenous genetic elements in a manner analogous to RNAi in eukaryotic organisms. Type II CRISPR-Cas systems can be engineered to direct targeted double-stranded DNA breaks in vitro to specific sequences. RNA-guided nucleases (RGNs) typically include two components: a short ˜100 nucleotide single guide RNA (gRNA), containing 20 variable nucleotides at the 5′ end involved in base pairing with a target DNA sequence, and the Cas9 nuclease, which cleaves the target DNA (Jinek, M., et al. Science 337, 816-821 (2012)).

The specificity of CRISPR-Cas is dictated by the identity of spacer sequences flanked by direct repeats encoded in the CRISPR locus, which are transcribed and processed into mature guide RNAs (gRNA) (Jinek, M. et al. (2012)). With the aid of a trans-activating small RNA (tracrRNA), gRNAs license the Cas9 endonuclease to introduce double-stranded breaks in target DNA sequences (protospacers) (Jinek, M. et al. (2012); Bikard, D., et al. (2012)). Thus, through simple modifications of spacers in the CRISPR loci, an RNA-guided nuclease can direct cleavage of almost any DNA sequence with the only design restriction being a NGG motif immediately 3′ of the protospacer (Jinek, M. et al. (2012)).

Non-limiting examples of RNA-guided nucleases that may be used herein include Cas9 and Cpf1.

Other programmable nucleases (and systems) may be used herein, including zinc-finger nucleases (ZFNs) and transcription activator-like effector nucleases (TALENs).

### Counterselectable Markers

In some embodiments, the engineered genetic constructs further comprise a nucleotide sequence encoding a counterselectable marker. A counterselectable marker is a molecule that promotes death of the cell harboring it (see, e.g., Reyrat, J. et al., Infect Immun. 66(9): 4011-4017, 1998). Cells that have integrated an engineered genetic construct that includes counterselectable marker, for example, are eliminated in the presence of a counterselective compound. Thus, counterselectable markers can be used, as provided herein, for the positive selection of cells that have undergone an excision/degradation (eraser) event leading to the loss of the counterselectable marker. In some embodiments, the cassette of an engineered genetic construct includes a (at least one) counterselectable marker, thus the counterselectable marker may be located between cognate recombinase recognition sites and/or may include nuclease recognition sites. In some embodiments, a counterselectable marker is downstream from a nucleic acid encoding a product of interest. In some embodiments, the promoter operably linked to the nucleotide sequence encoding a product of interest is also operably linked to the nucleotide sequence encoding the counterselectable marker.

In some embodiments, the counterselectable marker is a prodrug. A prodrug is a medication or compound that, after administration, is metabolized into a pharmacologically active drug. In some embodiments, the counterselectable marker is a cytosine deaminase. Cytosine deaminase converts 5-fluorocytosine (5-FC) into 5-fluorouracil (5-FU), which can result in cell toxicity. In some embodiments, the counterselectable marker is a thymidine kinases (e.g., HSV thymidine kinase). HSV thymidine kinase (HSV-tk) converts ganciclovir into a toxic product and can be used to trigger cell killing. Other counterselectable markers (e.g., kill switches) are known and may be used as provided herein.

Other counterselectable markers are encompassed by the present disclosure including, for example, those described by the following: Deans, T. L. et al. Cell 130, 363-372, 2007; Ramos, C. A et al. Stem Cells, 28(6): 1107-1115, 2010; and Chung, H. K. et al. Nature Chemical Biology, 11: 713-720, each of which is incorporated herein by reference).

### Engineered Nucleic Acids

An engineered nucleic acid (e.g., an engineered genetic construct) is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. The term “engineered nucleic acids” includes recombinant nucleic acids and synthetic nucleic acids. A “recombinant nucleic acid” refers to a molecule that is constructed by joining nucleic acid molecules and, in some embodiments, can replicate in a live cell. A “synthetic nucleic acid” refers to a molecule that is amplified or chemically, or by other means, synthesized. Synthetic nucleic acids include those that are chemically modified, or otherwise modified, but can base pair with naturally-occurring nucleic acid molecules. Recombinant nucleic acids and synthetic nucleic acids also include those molecules that result from the replication of either of the foregoing. Engineered nucleic acid of the present disclosure may be encoded by a single molecule (e.g., included in the same plasmid or other vector) or by multiple different molecules (e.g., multiple different independently-replicating molecules).

Engineered nucleic acid of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the ‘Y extension activity of a DNA polymerase and DNA ligase activity. The 5’ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, engineered nucleic acid constructs are produced using IN-FUSION® cloning (Clontech).

A promoter refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof. A promoter drives expression or drives transcription of the nucleic acid sequence that it regulates. Herein, a promoter is considered to be operably linked when it is in a correct functional location and orientation in relation to a nucleic acid sequence it regulates to control (“drive”) transcriptional initiation and/or expression of that sequence.

Constitutive promoters are unregulated promoters that continually activate transcription. Non-limiting examples of constitutive promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter.

Inducible promoters are promoters that are characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter. A promoter is considered responsive to a signal if in the presence of that signal transcription from the promoter is activated, deactivated, increased or decreased.

In some embodiments, a terminator sequence separates nucleotide sequences encoding recombinases and/or nucleases from downstream nucleotide sequences encoding products of interest. A terminator sequence is a nucleic acid sequence that causes transcription to stop. A terminator may be unidirectional or bidirectional. It is comprised of a DNA sequence involved in specific termination of an RNA transcript by an RNA polymerase. A terminator sequence prevents transcriptional activation of downstream nucleic acid sequences by upstream promoters. The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

Also provided herein are vectors comprising the engineered genetic constructs of the present disclosure. In some embodiments, the vector is an episomal vector, such as a plasmid or viral vector (e.g., adenoviral vector, retroviral vector, herpes simplex virus vectors, and/or chimeric viral vectors).

### Products of Interest

Products encoded by the engineered genetic constructs of the present disclosure may be, for example, therapeutic molecules and/or prophylactic molecules. In some embodiments, the product of interest is protein or peptide (e.g., a therapeutic protein or peptide). In some embodiments, the product of interest is a nucleic acid (e.g., a therapeutic nucleic acid). Examples of nucleic acids include RNA, DNA or a combination of RNA and DNA. In some embodiments the product interest is DNA (e.g., single-stranded DNA or double-stranded DNA). In some embodiments, the product of interest is RNA. For example, the product of interest may be selected form RNA interference (RNAi) molecules, such as short-hairpin RNAs, short interfering RNAs and micro RNAs.

Examples of therapeutic and/or prophylactic molecules, such as antibodies, enzymes, hormones, inflammatory agents, anti-inflammatory agents, immunomodulatory agents, and anti-cancer agents.

### Cells

The present disclosure provides, in some embodiments, cells comprising the engineered genetic constructs described herein and/or vectors containing the engineered genetic constructs described herein.

In some embodiments, the cell is a stem cell. For example, the stem cell may be a mesenchymal stem cell, a hematopoietic stem cell, an embryonic stem cell, or a pluripotent stem cell (e.g., induced pluripotent stem cell). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

In some embodiments, the cell is an immune cell. Non-limiting examples of immune cells that may be used as provided herein include natural killer (NK) cells, NKT cells, mast cells, eosinophils, basophils, macrophages, neutrophils, dendritic cells, T cells and B cells. Examples of T cells include, but are not limited to, CD8+ T cells, CD4+ T cells, gamma-delta T cells, and T regulatory cells (e.g., (CD4+, FOXP3+, CD25+ cells). In some embodiments, the T cell is a chimeric antigen receptor (CAR) T cells (e.g., fusions of single-chain variable fragments (scFv) derived from monoclonal antibodies, fused to CD3-zeta transmembrane- and endodomain).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

### Compositions and Kits

Also provided herein are compositions comprising at least one of the engineered genetic constructs of the present disclosure, a vector comprising at least one of the engineered genetic constructs of the present disclosure, or a cell comprising at least one of the engineered genetic constructs of the present disclosure.

Further provided herein are kits comprising at least one of the engineered genetic constructs of the present disclosure or at least one vector comprising at least one of the engineered genetic constructs of the present disclosure. The kits, in some embodiments, further comprise at least one inducer agent that modulates activity of the inducible promoter(s) of the expression cassette. In some embodiments, the kits further comprise a counterselective agent.

### Methods

Aspects of the present disclosure provide methods that include introducing into a population of cells at least one engineered genetic construct (e.g., encoding (a) at least one recombinase and/or at least one nuclease and (b) a product of interest and at least one counterselectable marker), wherein the product of interest aids in differentiation, expansion or phenotypic maintenance (persistence) of the cells (e.g., transcription factors, growth factors, etc.). In some embodiments, the methods further comprise culturing cells of the population and producing the product of interest. In some embodiments, the methods further comprise culturing cells of the population in the presence of an inducer agent, activating the inducible promoter operably linked to a recombinase(s), and excising the cassette from the engineered genetic construct (and/or excising heterologous nucleic acid in the cell). In some embodiments, the methods further comprise culturing cells of the population in the presence of a counterselective agent and killing cells that express the counterselectable marker. In some embodiments, the methods further comprise delivering cells of the population to a subject (e.g., a human subject).

In some embodiments, less than 20% of the cells of the population comprise the cassette following the step of culturing cells of the population in the presence of a counterselective agent. In some embodiments, less than 15% of the cells of the population comprise the cassette following the step of culturing cells of the population in the presence of a counterselective agent. In some embodiments, less than 10% of the cells of the population comprise the cassette following the step of culturing cells of the population in the presence of a counterselective agent. In some embodiments, less than 5% of the cells of the population comprise the cassette following the step of culturing cells of the population in the presence of a counterselective agent.

A population of cells (e.g., stem cells or immune cells) may include 10⁵-10¹¹ cells. For example, a population of cells may include 10⁵, 10⁶, 10⁷, 10⁸, 10⁹, 10¹⁰, or 10¹¹ cells. A population of cells, in some embodiments, is a homogeneous population of cells (all the same cell type), while in other embodiments, the population of cells is a heterogeneous population of cells (a mixture of different cell types).

Other aspects of the present disclosure provide introducing into a population of cells at least one engineered genetic construct (e.g., encoding (a) at least one recombinase and/or at least one nuclease and (b) a product of interest and at least one counterselectable marker), wherein the product of interest is a therapeutic molecule and/or a prophylactic molecule. In some embodiments, the methods further comprise delivering cells of the population to a subject. In some embodiments, the methods further comprise exposing the subject to an inducer agent to activate the inducible promoter and express the recombinase(s) and excising the cassette from the engineered genetic construct (and/or excising heterologous nucleic acid in the cell). In some embodiments, the methods further comprise exposing the subject to a counterselective agent and killing cells that express the counterselectable marker.

In some embodiments, less than 20% of the cells of the population comprise the cassette following the step of exposing the subject to a counterselective agent. In some embodiments, less than 15% of the cells of the population comprise the cassette following the step of exposing the subject to a counterselective agent. In some embodiments, less than 10% of the cells of the population comprise the cassette following the step of exposing the subject to a counterselective agent. In some embodiments, less than 5% (e.g., 4%, 3%, 2% or 1%) of the cells of the population comprise the cassette following the step of exposing the subject to a counterselective agent.

According to various aspects of the present disclosure, methods may include delivering to a cell any of the engineered constructs of the present disclosure. Likewise, any of the cells, as provided herein may be delivered to a subject, such as a human subject.

Engineered genetic constucts may be delivered to cells using a viral delivery system (e.g., retroviral, adenoviral, adeno-association, helper-dependent adenoviral systems, hybrid adenoviral systems, herpes simplex, pox virus, lentivirus, Epstein-Barr virus) or a non-viral delivery system (e.g., physical: naked DNA, DNA bombardment, electroporation, hydrodynamic, ultrasound or magnetofection; or chemical: cationic lipids, different cationic polymers or lipid polymer) (Nayerossadat N et al. Adv Biomed Res. 2012; 1: 27, incorporated herein by reference). In some embodiments, the non-viral based deliver system is a hydrogel-based delivery system (see, e.g., Brandl F, et al. Journal of Controlled Release, 2010, 142(2): 221-228, incorporated herein by reference).

Engineered genetic constructs and/or cells may be delivered to a subject (e.g., a mammalian subject, such as a human subject) by any in vivo delivery method known in the art. For example, engineered genetic constructs and/or cells may be delivered intravenously. In some embodiments, engineered genetic constructs and/or cells are delivered in a delivery vehicle (e.g., non-liposomal nanoparticle or liposome). In some embodiments, engineered genetic constructs and/or cells are delivered systemically to a subject having a cancer or other disease and activated (transcription is activated) specifically in cancer cells or diseased cells of the subject.

### Additional Embodiments

1. An engineered genetic construct comprising a cassette that comprises:

(a) an inducible promoter operably linked to a nucleotide sequence encoding a recombinase; and

(b) a promoter operably linked to a nucleotide sequence encoding a product of interest,

wherein the cassette is flanked by cognate recombinase recognition sites.

2. The engineered genetic construct of paragraph 1, wherein (a) is upstream from (b).
3. The engineered genetic construct of paragraph 2, wherein a terminator sequence is located between (a) and (b).
4. The engineered genetic construct of any one of paragraphs 1-3, wherein the recombinase is selected from tyrosine recombinases or tyrosine integrases.
5. The engineered genetic construct of paragraph 4, wherein the recombinase is selected from Cre, Dre, Flp, KD, B2, B3, λ, HK022 and HP1 recombinases.
6. The engineered genetic construct of any one of paragraphs 1-3, wherein the recombinase is selected from serine recombinases or serine integrases.
7. The engineered genetic construct of paragraph 6, wherein the recombinase is selected from γδ, ParA, Tn3, Gin, ΦC31, Bxb1 and R4 recombinases.
8. The engineered genetic construct of any one of paragraphs 1-7, wherein the product of interest is a therapeutic molecule or a prophylactic molecule.
9. The engineered genetic construct of any one of paragraphs 1-8, wherein the product of interest is a protein or peptide.
10. The engineered genetic construct of any one of paragraphs 1-9, wherein the product of interest is a nucleic acid.
11. The engineered genetic construct of paragraph 10, wherein the nucleic acid comprises RNA, DNA or a combination of RNA and DNA.
12. The engineered genetic construct of paragraph 10, wherein the RNA is selected from short-hairpin RNAs, short interfering RNAs and micro RNAs.
13. The engineered genetic construct of any one of paragraphs 1-12 further comprising (c) a nucleotide sequence encoding counterselectable marker.
14. The engineered genetic construct of paragraph 13, wherein the counterselectable marker is located between the cognate recombinase recognition sites.
15. The engineered genetic construct of paragraph 13 or 14, wherein the nucleotide sequence of (c) is downstream from the nucleotide sequence of (b).
16. The engineered genetic construct of paragraph 9 or 10, wherein the counterselectable marker is a prodrug.
17. The engineered genetic construct of any one of paragraphs 13-16, wherein the counterselectable marker is selected from cytosine deaminases and thymidine kinases.
18. The engineered genetic construct of any one of paragraphs 1-17, wherein the construct comprises at least two inducible promoters, each linked to a nucleotide sequence encoding a different recombinase, and wherein the cassette is flanked by recombinase recognition sites cognate to the different recombinases.
19. The engineered genetic construct of any one of paragraphs 1-17, wherein the inducible promoter is linked to at least two nucleotide sequences, each encoding a different recombinase, and wherein the cassette is flanked by recombinase recognition sites cognate to the different recombinases.
20. The engineered genetic construct of any one of paragraphs 1-17, wherein the construct comprises at least three inducible promoters, each linked to a nucleotide sequence encoding a different recombinase, and wherein the cassette is flanked by recombinase recognition sites cognate to the different recombinases.
21. The engineered genetic construct of any one of paragraphs 1-17, wherein the inducible promoter is linked to at least three nucleotide sequences, each encoding a different recombinase, and wherein the cassette is flanked by recombinase recognition sites cognate to the different recombinases.
22. An engineered genetic construct comprising a cassette that comprises:

(a) at least two inducible promoters, each operably linked to a nucleotide sequence encoding a different recombinase; and

(b) a promoter operably linked to a nucleotide sequence encoding a product of interest and a counterselectable molecule,

wherein the cassette is flanked by recombinase recognition sites cognate to the different recombinases.

23. A vector comprising the engineered genetic construct of any one of paragraphs 1-22.
24. The vector of paragraph 23, wherein the vector is a plasmid or a viral vector.
25. A cell comprising the engineered genetic construct of any one of paragraphs 1-22.
26. A cell comprising the vector of paragraph 23 or 24.
27. The cell of paragraph 25 or 26, wherein the cell is a stem cell.
28. The cell of paragraph 27, wherein the stem cell is selected from mesenchymal stem cells, hematopoietic stem cells, embryonic stem cells, and pluripotent stem cells.
29. The cell of paragraph 25 or 26, wherein the cell is an immune cell.
30. The cell of paragraph 29, wherein the immune cell is selected from natural killer (NK) cells, NKT cells, mast cells, eosinophils, basophils, macrophages, neutrophils, dendritic cells, T cells and B cells.
31. The cell of paragraph 30, wherein the T cell is selected from CD8+ T cells, CD4+ T cells, gamma-delta T cells, and T regulatory cells.
32. The cell of paragraph 30 or 31, wherein the T cell is a chimeric antigen receptor (CAR) T cell or an engineered T cell receptor (TCR) cell.
33. A composition comprising the engineered genetic construct of any one of paragraphs 1-22.
34. A composition comprising the vector of paragraph 23 or 24.
35. A composition comprising the cell of any one of paragraphs 25-32.
36. A kit comprising the engineered genetic construct of any one of paragraphs 1-22 and at least one inducer agent that modulates activity of the inducible promoter(s) of (a).
37. A kit comprising the vector of paragraph 23 or 24 and at least one inducer agent that modulates activity of the inducible promoter(s) of (a).
38. A method comprising introducing into a cell the engineered genetic construct of any one of paragraphs 1-24, wherein the product of interest aids in differentiation, expansion or phenotypic maintenance (persistence) of the cell.
39. The method of paragraph 38 wherein the promoter of (b) is a constitutive promoter.
40. The method of paragraph 38 or 39 further comprising culturing the cell and producing the product of interest.
41. The method of any one of paragraphs 38-40 further comprising activating the promoter of (a), expressing the recombinase and excising heterologous nucleic acid in the cell.
42. The method of paragraph 41 further comprising delivering the cell to a subject.
43. A method comprising introducing into a cell the engineered genetic construct of any one of paragraphs 1-24, wherein the product of interest is a therapeutic or prophylactic molecule.
44. The method of paragraph 43 wherein the promoter of (b) is a constitutive promoter.
45. The method of paragraph 43 or 44 further delivering the cell to a subject.
46. The method of paragraph 45 further comprising activating the promoter of (a), expressing the recombinase and excising heterologous nucleic acid in the cell.
47. A method comprising delivering to a subject the cell of any one of paragraphs 25-32.
48. An engineered genetic construct comprising a cassette that comprises:

(a) an inducible promoter operably linked to a nucleotide sequence encoding a nuclease; and

(b) a promoter operably linked to a nucleotide sequence encoding a product of interest,

wherein the engineered genetic construct comprises cognate nuclease recognition sites, optionally flanking the cassette.

49. The engineered genetic construct of paragraph 48, wherein (a) is upstream from (b).
50. The engineered genetic construct of paragraph 49, wherein a terminator sequence is located between (a) and (b).
51. The engineered genetic construct of any one of paragraphs 48-50, wherein the nuclease is selected from meganucleases and RNA-guided nucleases.
52. The engineered genetic construct of paragraph 51, wherein the meganucleases are selected from intron endonucleases and intein endonucleases.
53. The engineered genetic construct of paragraph 51, wherein the RNA-guided nucleases are selected from Cas9 nucleases and Cpf1 nucleases.
54. The engineered genetic construct of paragraph D6, further comprising (c) nucleotide sequences encoding guide RNAs (gRNAs) complementary to the nuclease recognitions sites and located between the nuclease recognition sites.
55. The engineered genetic construct of any one of paragraphs 48-54, wherein the product of interest is a therapeutic molecule or a prophylactic molecule.
56. The engineered genetic construct of any one of paragraphs 48-55, wherein the product of interest is a protein or peptide.
57. The engineered genetic construct of any one of paragraphs 48-56, wherein the product of interest is a nucleic acid.
58. The engineered genetic construct of paragraph 57, wherein the nucleic acid comprises RNA, DNA or a combination of RNA and DNA.
59. The engineered genetic construct of paragraph 57, wherein the RNA is selected from short-hairpin RNAs, short interfering RNAs and micro RNAs.
60. The engineered genetic construct of any one of paragraphs 48-59 further comprising (c) a nucleotide sequence encoding counterselectable marker.
61. The engineered genetic construct of paragraph 60, wherein the counterselectable marker is located between the cognate nuclease recognition sites.
62. The engineered genetic construct of paragraph 60 or 61, wherein the nucleotide sequence of (c) is downstream from the nucleotide sequence of (b).
63. The engineered genetic construct of paragraph 56 or 57, wherein the counterselectable marker is a prodrug.
64. The engineered genetic construct of any one of paragraphs 60-63, wherein the counterselectable marker is selected from cytosine deaminases and thymidine kinases.
65. The engineered genetic construct of any one of paragraphs 48-64, wherein the construct comprises at least two inducible promoters, each linked to a nucleotide sequence encoding a different nuclease, and wherein the engineered genetic construct comprises cognate nuclease recognition sites, optionally flanking the cassette.
66. The engineered genetic construct of any one of paragraphs 48-64, wherein the inducible promoter is linked to at least two nucleotide sequences, each encoding a different nuclease, and wherein the engineered genetic construct comprises cognate nuclease recognition sites, optionally flanking the cassette.
67. The engineered genetic construct of any one of paragraphs 48-64, wherein the construct comprises at least three inducible promoters, each linked to a nucleotide sequence encoding a different nuclease, and wherein the engineered genetic construct comprises cognate nuclease recognition sites, optionally flanking the cassette.
68. The engineered genetic construct of any one of paragraphs 48-64, wherein the inducible promoter is linked to at least three nucleotide sequences, each encoding a different nuclease, and wherein the engineered genetic construct comprises cognate nuclease recognition sites, optionally flanking the cassette.
69. An engineered genetic construct comprising a cassette that comprises:

(a) at least two inducible promoters, each operably linked to a nucleotide sequence encoding a different nuclease; and

(b) a promoter operably linked to a nucleotide sequence encoding a product of interest and a counterselectable molecule,

wherein the cassette is flanked by nuclease recognition sites cognate to the different nucleases.

70. A vector comprising the engineered genetic construct of any one of paragraphs 48-69.
71. The vector of paragraph 70, wherein the vector is a plasmid or a viral vector.
72. A cell comprising the engineered genetic construct of any one of paragraphs 48-69.
73. A cell comprising the vector of paragraph 70 or 71.
74. The cell of paragraph 72 or 73, wherein the cell is a stem cell.
75. The cell of paragraph 74, wherein the stem cell is selected from mesenchymal stem cells, embryonic stem cells, and pluripotent stem cells.
76. The cell of paragraph 72 or 73, wherein the cell is an immune cell.
77. The cell of paragraph 76, wherein the immune cell is selected from natural killer (NK) cells, NKT cells, mast cells, eosinophils, basophils, macrophages, neutrophils, dendritic cells, T cells and B cells.
78. The cell of paragraph 77, wherein the T cell is selected from CD8+ T cells, CD4+ T cells, gamma-delta T cells, and T regulatory cells.
79. The cell of paragraph 77 or 78, wherein the T cell is a chimeric antigen receptor (CAR) T cell or an engineered T cell receptor (TCR) cell.
80. A composition comprising the engineered genetic construct of any one of paragraphs D1-D22.
81. A composition comprising the vector of paragraph 70 or 71.
82. A composition comprising the cell of any one of paragraphs 72-79.
83. A kit comprising the engineered genetic construct of any one of paragraphs 48-69 and at least one inducer agent that modulates activity of the inducible promoter(s) of (a).
84. A kit comprising the vector of paragraph 70 or 71 and at least one inducer agent that modulates activity of the inducible promoter(s) of (a).
85. A method comprising introducing into a cell the engineered genetic construct of any one of paragraphs 48-71, wherein the product of interest aids in differentiation, expansion or phenotypic maintenance (persistence) of the cell.
86. The method of paragraph 85 wherein the promoter of (b) is a constitutive promoter.
87. The method of paragraph 85 or 86 further comprising culturing the cell and producing the product of interest.
88. The method of any one of paragraphs 85-87 further comprising activating the promoter of (a), expressing the recombinase and excising heterologous nucleic acid in the cell.
89. The method of paragraph 88 further comprising delivering the cell to a subject.
90. A method comprising introducing into a cell the engineered genetic construct of any one of paragraphs 48-71, wherein the product of interest is a therapeutic or prophylactic molecule.
91. The method of paragraph 90 wherein the promoter of (b) is a constitutive promoter.
92. The method of paragraph 90 or 91 further delivering the cell to a subject.
93. The method of paragraph 92 further comprising activating the promoter of (a), expressing the recombinase and excising heterologous nucleic acid in the cell.
94. A method comprising delivering to a subject the cell of any one of paragraphs 72-79.
95. An engineered cell comprising:

(a) a nucleic acid comprising a promoter operably linked to a nucleotide sequence encoding a ligand-dependent chimeric recombinase; and

(b) a nucleic acid comprising a promoter operably linked to a nucleotide sequence encoding a product of interest, wherein the nucleic acid of (b) is flanked by cognate recombinase recognition sites.

96. The engineered cell of paragraph 95, wherein the recombinase is a ligand-dependent chimeric recombinase.
97. The engineered cell of paragraph 96, wherein the ligand-dependent chimeric recombinase is linked to a mutated human estrogen receptor (ER) ligand binding domain.
98. The engineered cell of any one of paragraphs 95-97, wherein the product of interest is a therapeutic molecule or a prophylactic molecule.
99. The engineered cell of any one of paragraphs 95-98, wherein the product of interest is a protein or peptide.
100. The engineered cell of any one of paragraphs 95-98, wherein the product of interest is a nucleic acid.
101. The engineered cell of paragraph 100, wherein the nucleic acid comprises RNA, DNA or a combination of RNA and DNA.
102. The engineered cell of paragraph 101, wherein the RNA is selected from short-hairpin RNAs, short interfering RNAs and micro RNAs.
103. The engineered cell of any one of paragraphs 95-102, wherein the nucleic acid of (b) further comprises a nucleotide sequence encoding counterselectable marker.
104. The engineered cell of paragraph 103, wherein the counterselectable marker is located between the cognate nuclease recognition sites.
105. The engineered cell of paragraph 103 or 104, wherein the nucleotide sequence encoding counterselectable marker is downstream from the nucleotide sequence of (b).
106. The engineered cell of any one of paragraphs 103-105, wherein the counterselectable marker is a prodrug.
107. The engineered cell of any one of paragraphs 103-105, wherein the counterselectable marker is selected from cytosine deaminases and thymidine kinases.
108. The engineered cell of any one of paragraphs 95-107, wherein the cell is a stem cell.
109. The engineered cell of paragraph 108, wherein the stem cell is selected from mesenchymal stem cells, embryonic stem cells, and pluripotent stem cells.
110. The engineered cell of any one of paragraphs 95-107, wherein the cell is an immune cell.
111. The engineered cell of paragraph 110, wherein the immune cell is selected from natural killer (NK) cells, NKT cells, mast cells, eosinophils, basophils, macrophages, neutrophils, dendritic cells, T cells and B cells.
112. The engineered cell of paragraph 111, wherein the T cell is selected from CD8+ T cells, CD4+ T cells, gamma-delta T cells, and T regulatory cells.
113. The engineered cell of paragraph 111 or 112, wherein the T cell is a chimeric antigen receptor (CAR) T cell or an engineered T cell receptor (TCR) cell.
114. A composition comprising the engineered cell of any one of paragraphs 95-113.
115. A kit comprising:

(a) a nucleic acid comprising a promoter operably linked to a nucleotide sequence encoding a ligand-dependent chimeric recombinase; and

(b) a nucleic acid comprising a promoter operably linked to a nucleotide sequence encoding a product of interest, wherein the nucleic acid of (b) is flanked by cognate recombinase recognition sites.

116. A method comprising culturing the engineered cell of any one of paragraphs 95-113 and producing the product of interest.
117. The method of paragraph 116, wherein the ligand-dependent chimeric recombinase is linked to a mutated human estrogen receptor (ER) ligand binding domain.
118. The method of paragraph 116 or 117 further comprising contacting the engineered cell with an inducer agent and excising heterologous nucleic acid in the cell.
119. The method of paragraph 118, wherein the inducer agent is 4-hydroxytamoxifen (OHT) and/or ICI 182,780 (ICI).
120. The method of paragraph 119 further comprising delivering the engineered cell to a subject.
121. An engineered cell comprising:

(a) a nucleic acid comprising a promoter operably linked to a nucleotide sequence encoding a first fragment of a recombinase;

(b) a nucleic acid comprising a promoter operably linked to a nucleotide sequence encoding a second fragment of a recombinase, wherein the first fragment and the second fragment when combined form a full-length functional recombinase; and

(c) a nucleic acid comprising a promoter operably linked to a nucleotide sequence encoding a product of interest, wherein the nucleic acid of (c) is flanked by cognate recombinase recognition sites.

122. The engineered cell of paragraph 121, wherein the first fragment is linked to a FKBP domain and the second fragment is linked to FRB domain.
123. The engineered cell of paragraph 121 or 122, wherein the product of interest is a therapeutic molecule or a prophylactic molecule.
124. The engineered cell of any one of paragraphs 121-123, wherein the product of interest is a protein or peptide.
125. The engineered cell of any one of paragraphs 121-123, wherein the product of interest is a nucleic acid.
126. The engineered cell of paragraph 125, wherein the nucleic acid comprises RNA, DNA or a combination of RNA and DNA.
127. The engineered cell of paragraph 126, wherein the RNA is selected from short-hairpin RNAs, short interfering RNAs and micro RNAs.
128. The engineered cell of any one of paragraphs 121-127, wherein the nucleic acid of (c) further comprises a nucleotide sequence encoding counterselectable marker.
129. The engineered cell of paragraph 128, wherein the counterselectable marker is located between the cognate nuclease recognition sites.
130. The engineered cell of paragraph 128 or 128, wherein the nucleotide sequence encoding counterselectable marker is downstream from the nucleotide sequence of (c).
131. The engineered cell of any one of paragraphs 128-130, wherein the counterselectable marker is a prodrug.
132. The engineered cell of any one of paragraphs 128-130, wherein the counterselectable marker is selected from cytosine deaminases and thymidine kinases.
133. The engineered cell of any one of paragraphs 121-132, wherein the cell is a stem cell.
134. The engineered cell of paragraph 133, wherein the stem cell is selected from mesenchymal stem cells, embryonic stem cells, and pluripotent stem cells.
135. The engineered cell of any one of paragraphs 121-132, wherein the cell is an immune cell.
136. The engineered cell of paragraph 135, wherein the immune cell is selected from natural killer (NK) cells, NKT cells, mast cells, eosinophils, basophils, macrophages, neutrophils, dendritic cells, T cells and B cells.
137. The engineered cell of paragraph 136, wherein the T cell is selected from CD8+ T cells, CD4+ T cells, gamma-delta T cells, and T regulatory cells.
138. The engineered cell of paragraph 136 or 137, wherein the T cell is a chimeric antigen receptor (CAR) T cell or an engineered T cell receptor (TCR) cell.
139. A composition comprising the engineered cell of any one of paragraphs 121-138.
140. A kit comprising:

(a) a nucleic acid comprising a promoter operably linked to a nucleotide sequence encoding a first fragment of a recombinase;

(b) a nucleic acid comprising a promoter operably linked to a nucleotide sequence encoding a second fragment of a recombinase, wherein the first fragment and the second fragment when combined form a full-length functional recombinase; and

(c) a nucleic acid comprising a promoter operably linked to a nucleotide sequence encoding a product of interest, wherein the nucleic acid of (c) is flanked by cognate recombinase recognition sites.

141. A method comprising culturing the engineered cell of any one of paragraphs 121-138 and producing the product of interest.
142. The method of paragraph 141, wherein the first fragment is linked to a FKBP domain and the second fragment is linked to FRB domain.
143. The method of paragraph 141 or 142 further comprising contacting the engineered cell with an inducer agent and excising heterologous nucleic acid in the cell.
144. The method of paragraph 143, wherein the inducer agent is 4-hydroxytamoxifen (OHT) and/or ICI 182,780 (ICI).
145. The method of paragraph 144 further comprising delivering the engineered cell to a subject.
146. An engineered genetic construct comprising

a cassette comprising (a) a promoter operably linked to a nucleotide sequence encoding a first product of interest upstream from (b) a promoter operably linked to a nucleotide sequence encoding a second product of interest and optionally a counterselectable marker, wherein expression or activity of the first product of interest is activatable (inducible), and wherein the first product of interest modulates excision or degradation of the cassette.

147. The engineered genetic construct of paragraph 146, wherein the promoter of (a) is an inducible promoter.
148. The engineered genetic construct of paragraph 146 or 147, wherein the cassette is flanked by recombinase recognition sites.
149. The engineered genetic construct of paragraph 148, wherein the first product of interest is a cognate recombinase.
150. The engineered genetic construct of paragraph 146 or 147, wherein the cassette is flanked by nuclease recognition sites.
151. The engineered genetic construct of paragraph 150, wherein the first product of interest is a cognate nuclease.
152. The engineered genetic construct of any one of paragraphs 146-151, wherein the second product of interest is a therapeutic molecule or a prophylactic molecule.
153. A cell comprising the engineered genetic construct of any one of paragraphs 146-152.
154. A composition or kit comprising the engineered genetic construct of any one of paragraphs 146-152or the cell of paragraph K8
155. A method of delivering to a subject the cell of paragraph 153.
156. An engineered genetic circuit comprising:

(a) one or more recombinases that are regulated by inducible transcription of recombinase genes, inducible translocation of recombinases into the nucleus, and/or inducible dimerization of split recombinases;

(b) one or more meganucleases or CRISPR-Cas nucleases; and/or

(c) one or more counter-selectable markers.

### EXAMPLES

### Example 1

In this Example, recombinase proteins are used to excise heterologous genetic cassettes (FIG. 1). Control of recombinase activity is achieved through inducible transcription of recombinase genes, inducible translocation of recombinases into the nucleus, and/or inducible dimerization of split recombinases. For example, recombinase expression is controlled through the use of doxycycline, phloretin (9), vanillic acid (13), macrolides (12), or other inducers. As another example, a recombinase can be fused to a nuclear receptor so the recombinase does not catalyze recombination in the absence of an inducer agent. Upon addition of an inducer agent, such as 4OHT, the recombinase translocates into the nucleus and catalyzes recombination (14, 15). This approach has been validated for Cre, Flp, and PhiC31 and is extensible to additional integrases (15-17). As yet another example, a recombinase protein can be split into two fragments that are inactive on their own, but can be dimerized by the addition of a small-molecule (e.g., rapamycin analogs) due to interactions with two protein domains, each fused to one of the fragments (e.g., FKBP and FRB) (18). This approach has been demonstrated for Cre and is extensible to other recombinases. Libraries of recombinases (19) can used in this manner.

### Example 2

In this Example, nuclease proteins, such as meganucleases or CRISPR-Cas nucleases, are used to excise or degrade heterologous genetic cassettes through targeted double-stranded breaks (FIG. 2). These nucleases are programmed to cut at specific sites that surround our heterologous DNA constructs, which has been shown to significantly enhance the efficiency of genetic deletion (20). The addition of donor DNA that is homologous to the flanking ends of region to be deleted can be used to enhance deletion efficiencies. These effectors are placed under the control of inducible promoters, in some embodiments.

### Example 3

In this Example, counterselectable markers, such as cytosine deaminase or thymidine kinase, are used to kill cells that still contain heterologous DNA following an excision or degradation reaction (see FIG. 3). For example, yeast or bacterial cytosine deaminase converts 5-fluorocytosine (5-FC) into 5-fluorouracil (5-FU), which can result in cell toxicity (21, 22). HSV thymidine kinase (HSV-tk) converts ganciclovir into a toxic product and can be used to trigger cell killing, although toxicity can be variable depending on cell background (23). An inducible kill switch may be used, which expresses the alpha chain of diphtheria toxin under the control of IPTG to trigger cell death (24). In some embodiments, the inducible activation of toxins through dimerization via small molecules can be used to enhance cell killing.

### Example 4

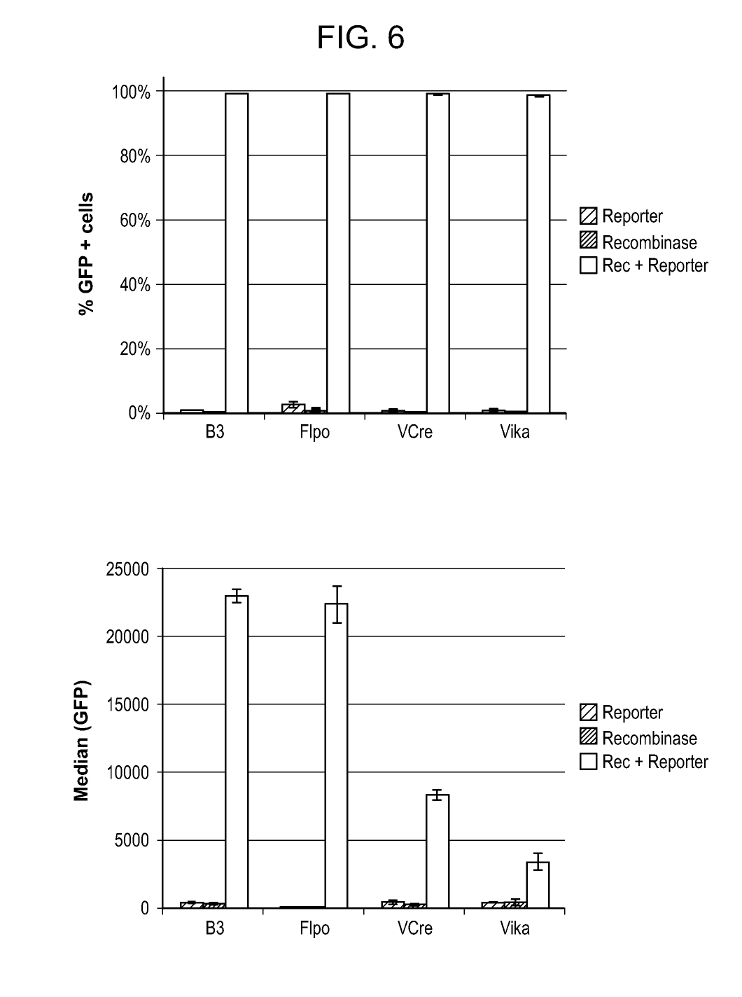

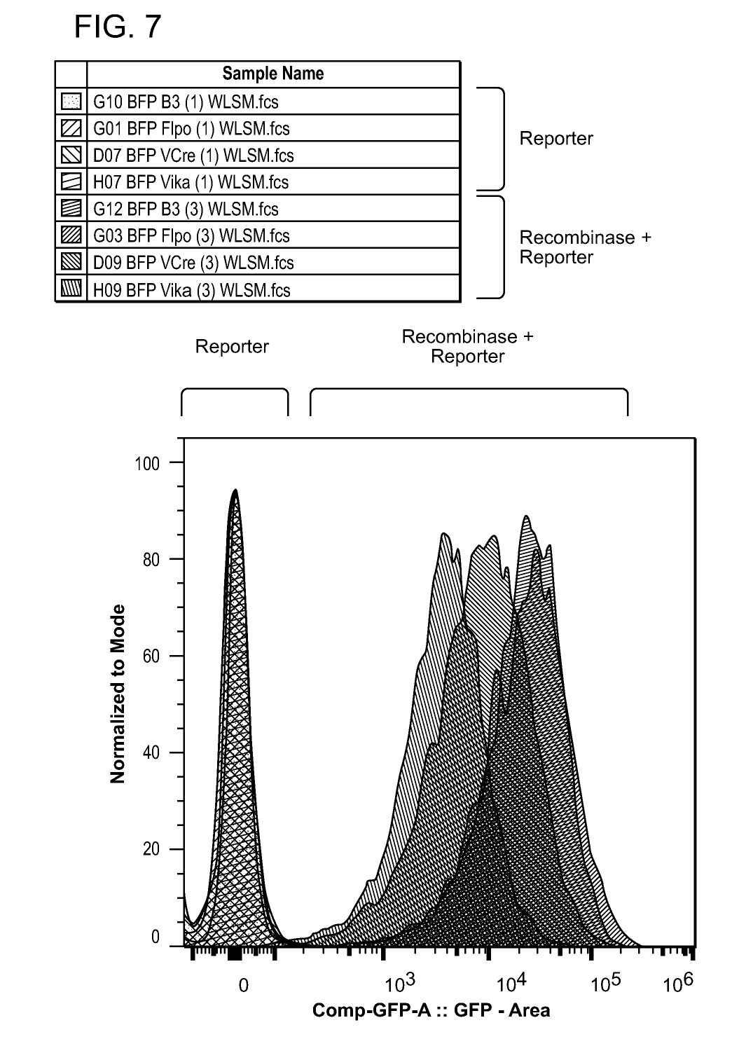

In this Example, transient 293FT cell transfection assays were performed to test recombinase activity of various tyrosine recombinases. 293FT cells were transiently transfected with an equal ratio of reporter plasmid, recombinase plasmid, and a transfection marker plasmid (e.g., a plasmid encoding BFP). The cells were assayed for GFP fluorescence 24 hours post transfection and gated for BFP expression. FIGS. 6 and 7 show data from the transient 293FT cell transfection assays. The top graph represents % GFP+ cells, and the bottom graph represents the median of the GFP mean fluorescence intensity of the GFP+ cells.

### Example 5

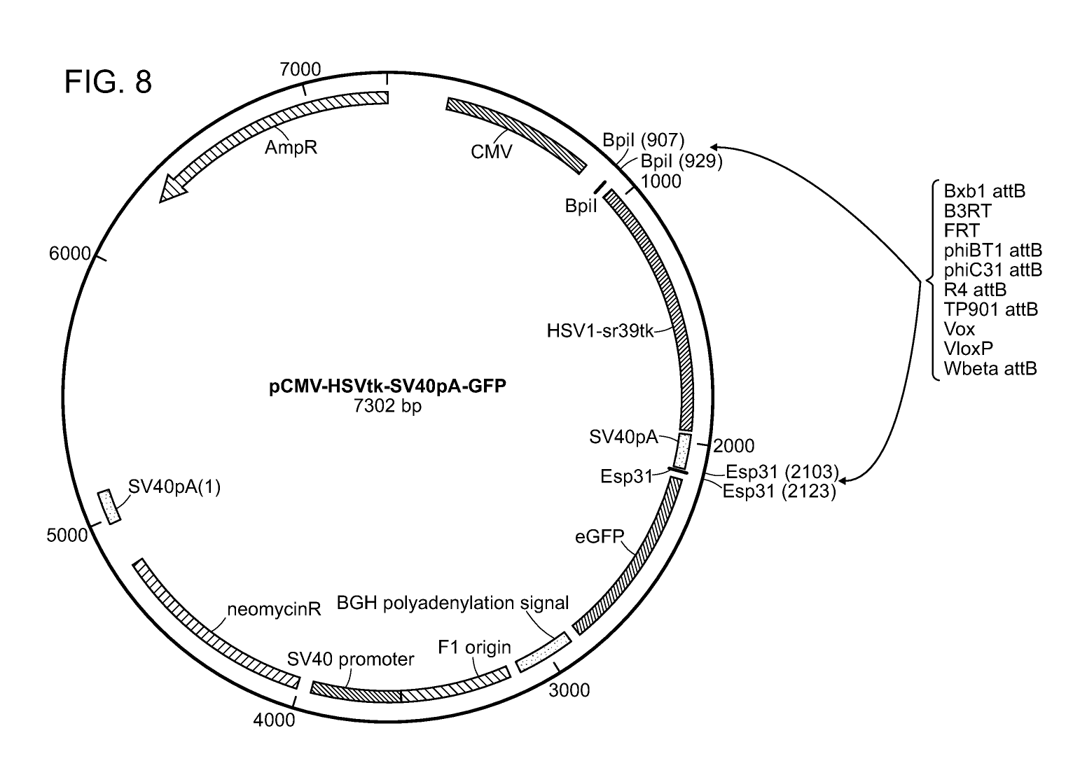

In this Example, a system was used to assay for recombinase excision efficiency. A BpiI(x2)-HSVtk-SV40 pA-EGFP-Esp3I(x2) cassette was inserted into the pcDNA3.1(+) mammalian expression vector (Life Tech). Recombination sequences were inserted into the BpiI and Esp3I sites via Golden Gate digestion/ligation. See FIG. 8.

### Example 6

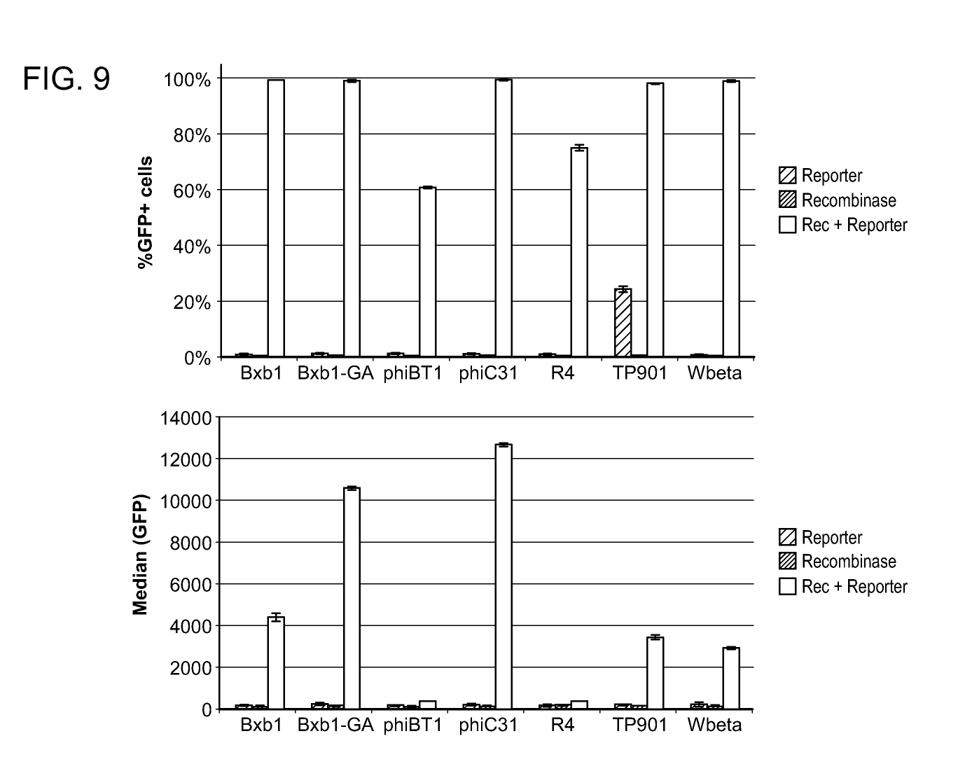

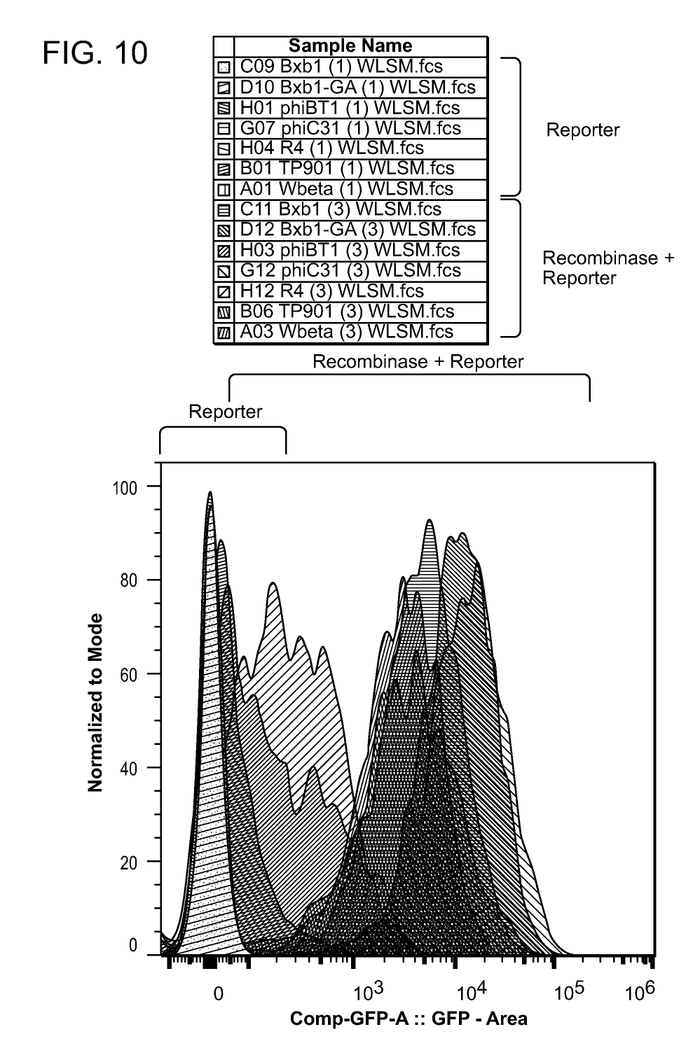

In this Example, transient 293FT cell transfection assays were performed to test recombinase activity of various serine integrases. 293FT cells were transiently transfected with an equal ratio of reporter plasmid, recombinase plasmid, and a transfection marker plasmid (e.g., a plasmid encoding BFP). The cells were assayed for GFP fluorescence 24 hours post transfection and gated for BFP expression. FIGS. 9 and 10 show data from the transient 293FT cell transfection assays. The top graph represents % GFP+ cells, and the bottom graph represents the median of the GFP mean fluorescence intensity of the GFP+ cells.

### Example 7

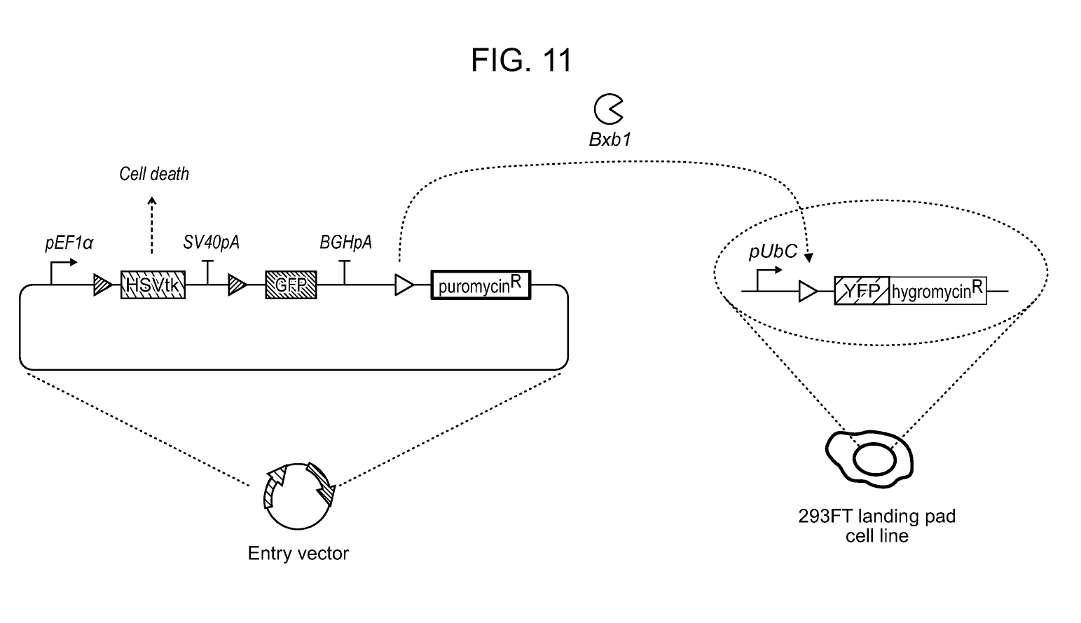

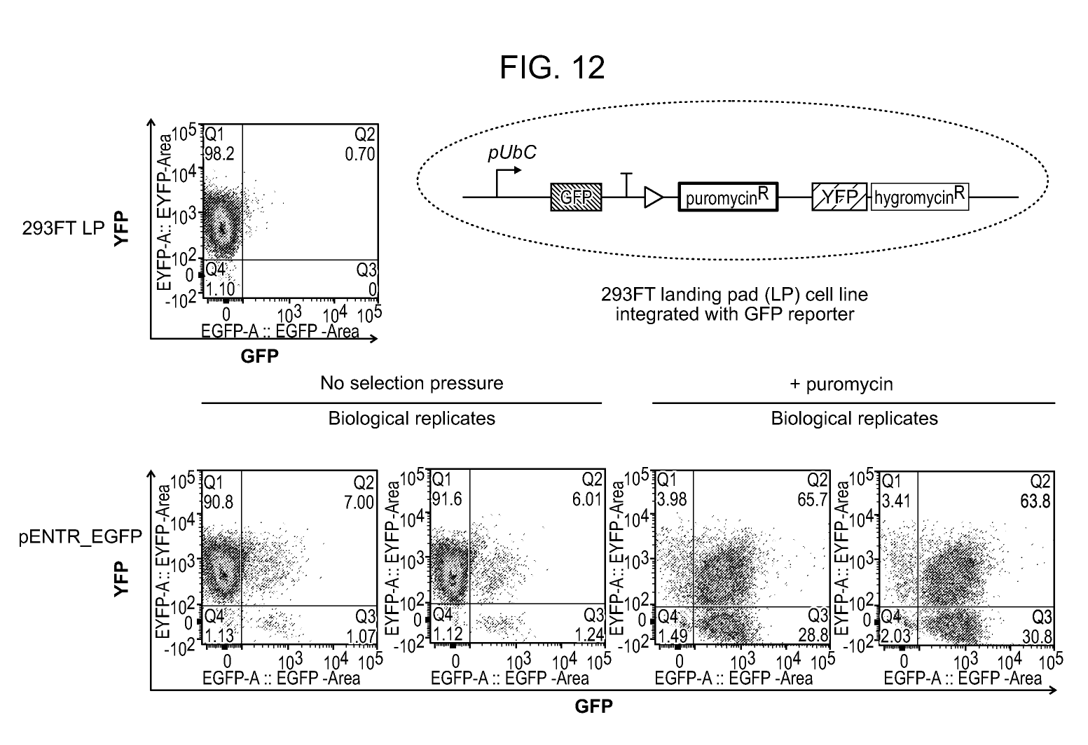

In this Example, an entry vector encoding a recombinase-based excision construct is integrated into a pre-engineered 293FT landing pad cell line that expresses YFP and hygromycin. See FIG. 11. FIG. 12 shows successful integration of a GFP reporter into the 293FT landing pad cell line. Upon integration, cells express GFP and simultaneously lose expression of YFP. Selection pressure with puromycin removes unintegrated cells.

### Example 8

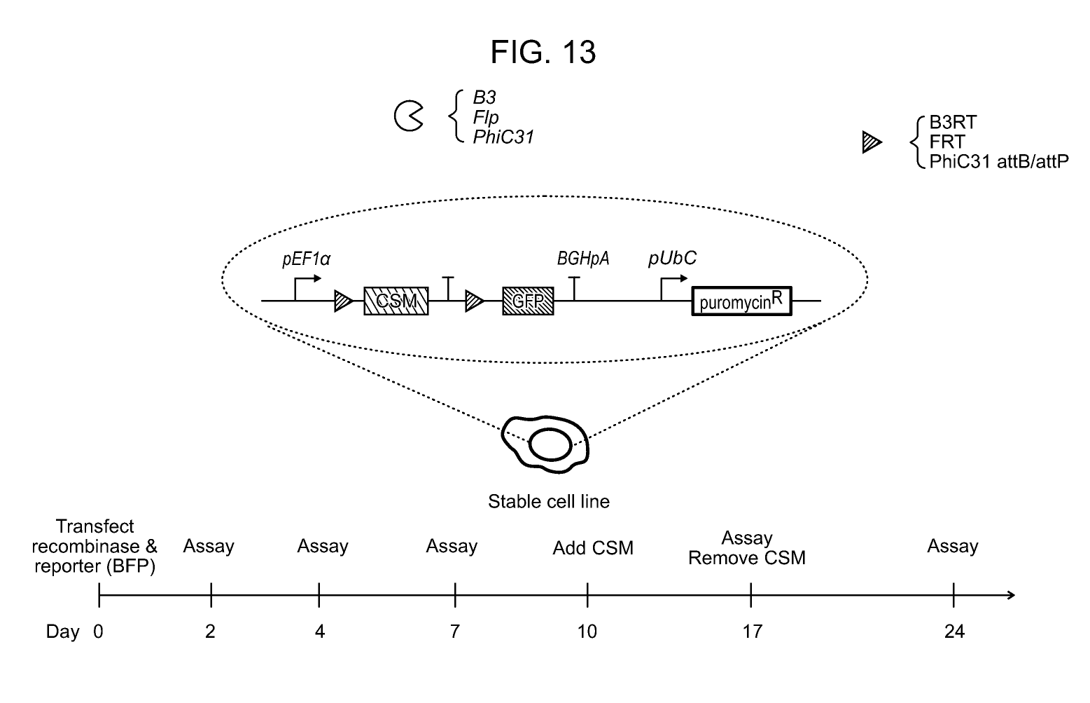

This Examples outlines a method for excising genomically-integrated constructs. An entry vector encoding a recombinase-based excision construct is integrated into a pre-engineered 293FT landing pad cell line. Integrated cell lines are transiently transfected with a recombinase-expressing plasmid and a reporter plasmid (e.g., expressing BFP) and assayed for GFP expression over time. A counter-selection marker (CSM) is used to kill off cells that retain the integrated construct. See FIG. 13.

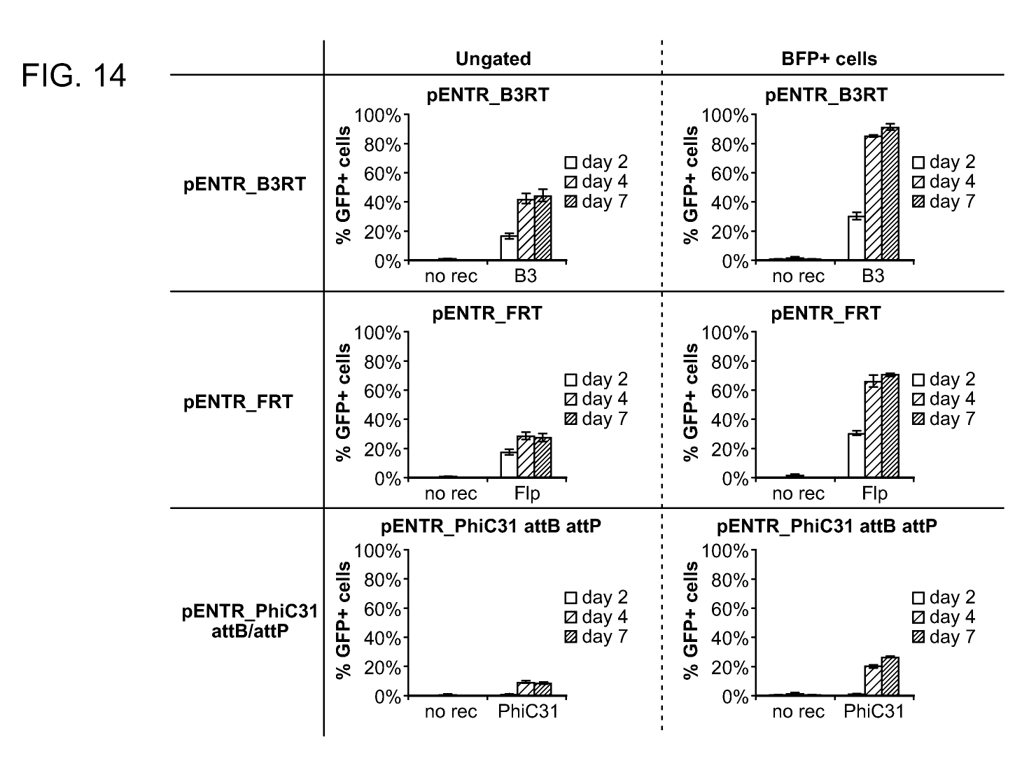

Cell lines expressing 3 different recombinase-based excision constructs were transiently transfected with the cognate recombinase, and GFP expression was assayed over time. See FIG. 14.

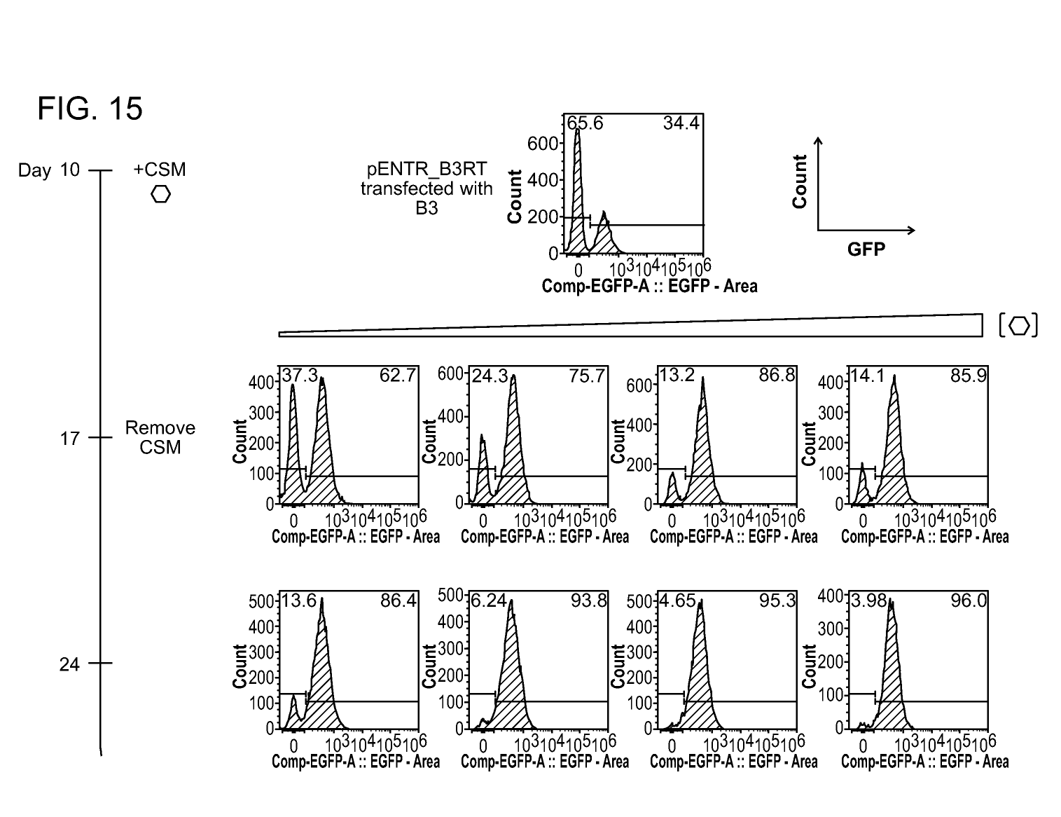

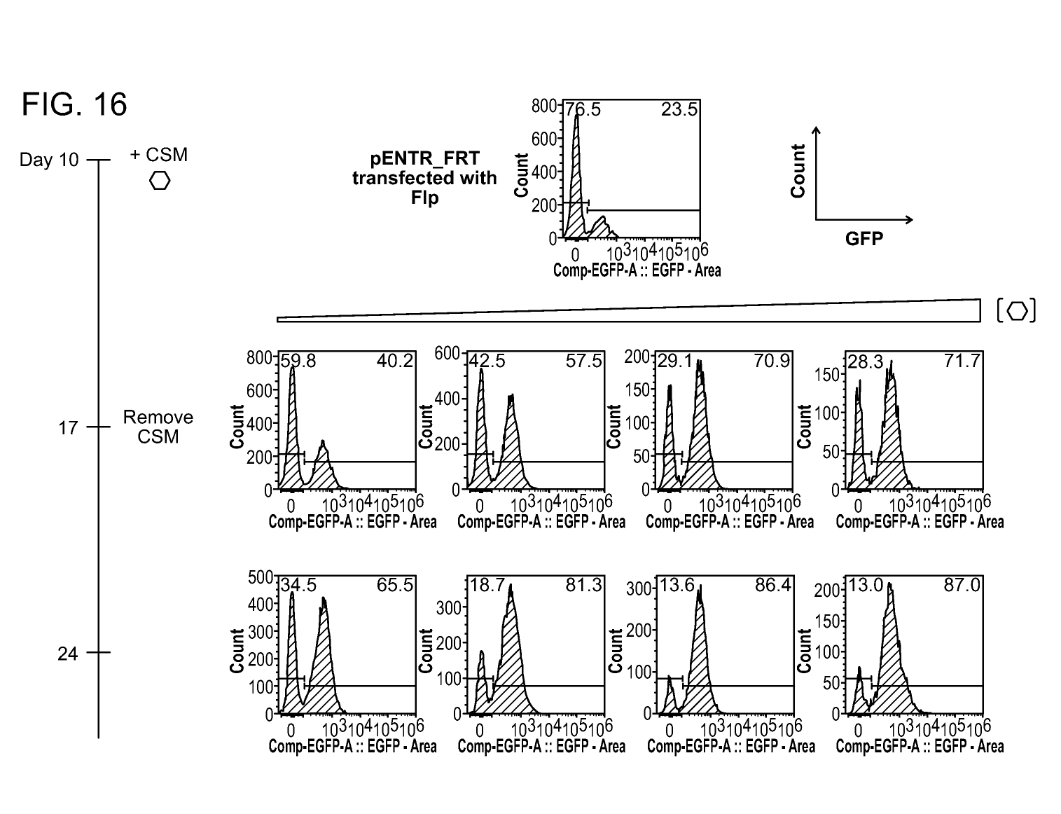

Following transient transfection of the B3 recombinase, a prodrug was applied to kill off cells that retained the pENTR_B3RT excision construct. The counter selection marker (CSM) converts the prodrug into a toxic drug. In this case, the CSM was HSVtk and the prodrug was ganciclovir (GCV). The cells were treated with 0.5, 1, 2, and 5 μM GCV for 7 days, and GFP expression was assayed over time. The histograms represent the % of GFP- and % of GFP+ cells. See FIG. 15. Following transient transfection of the Flp recombinase, GCV was applied, as above, to kill off cells that retained the pENTR_FRT excision construct. See FIG. 16.

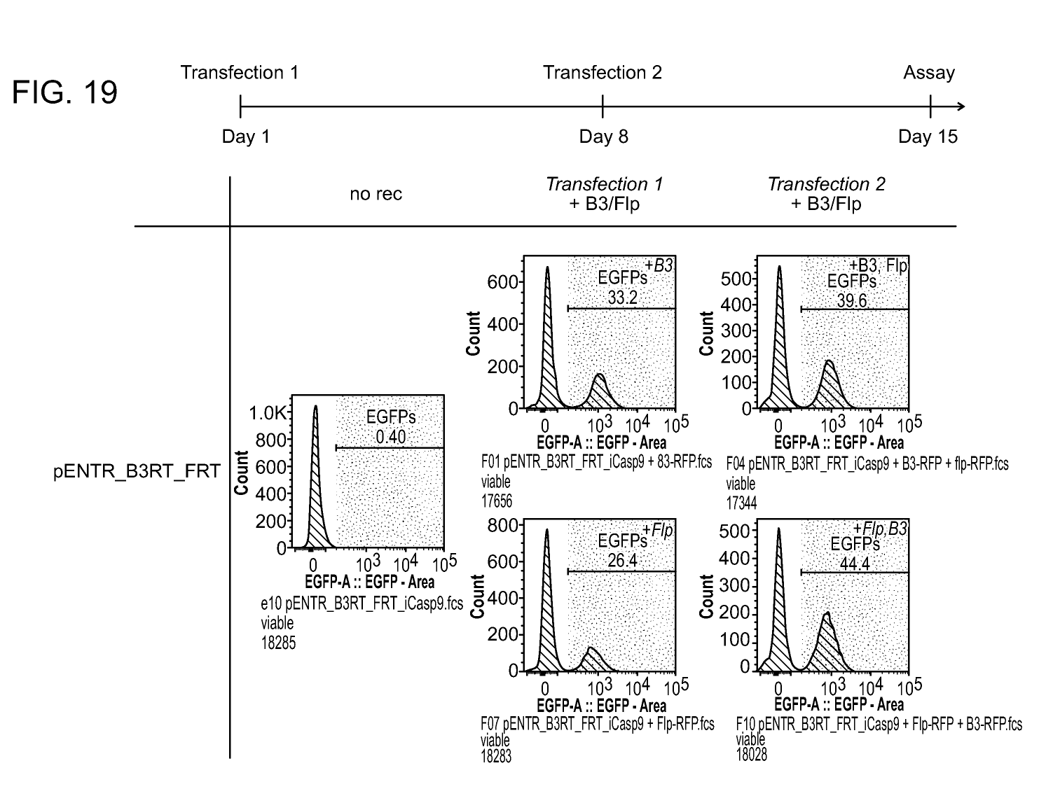

Cell lines expressing the pENTR_B3RT_FRT recombinase-based excision construct were sequentially transfected with B3 or Flp according to the indicated timeline. GFP expression was assayed over time, and the histograms represent the % of GFP+ cells on day 15. The pENTR_B3RT_FRT recombinase-based excision construct encodes the construct B3RT_FRT_iCasp9_SV40 pA_FRT_B3RT_EGFP_BGHpA, in which B3RT and FRT are recombination sequences for B3 and Flp, respectively; and iCasp9 is a counter selection marker (CSM). See FIG. 19

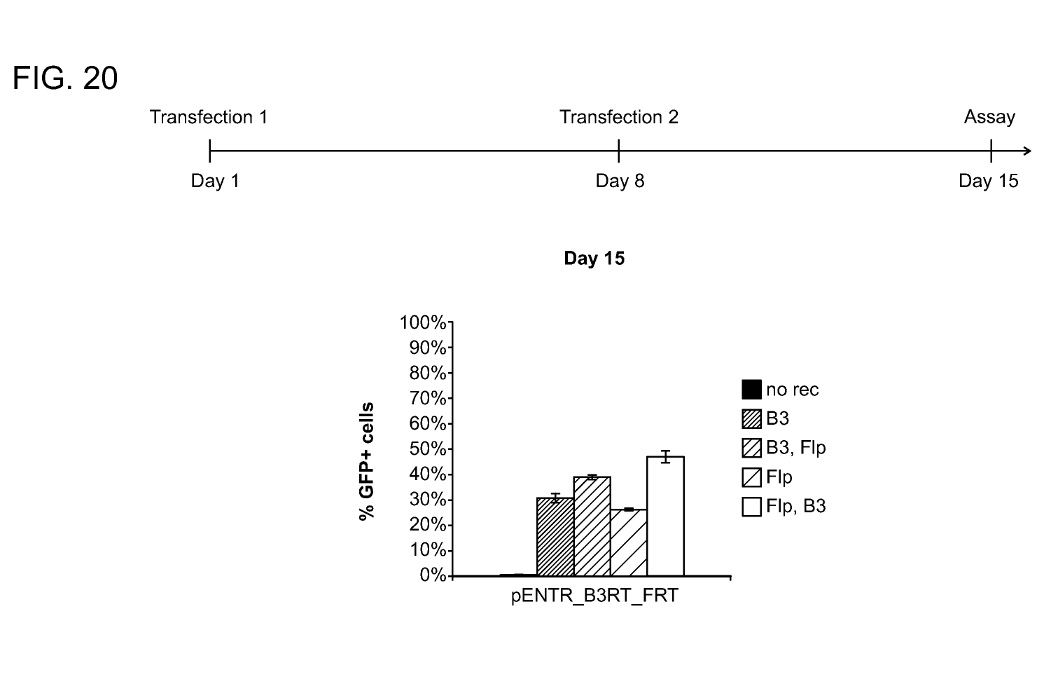

Cell lines expressing the pENTR_B3RT_FRT recombinase-based excision construct were sequentially transfected with B3 or Flp according to the indicated timeline. GFP expression was assayed over time, and the % of GFP+ cells on day 15 were plotted. See FIG. 20.

### Example 9

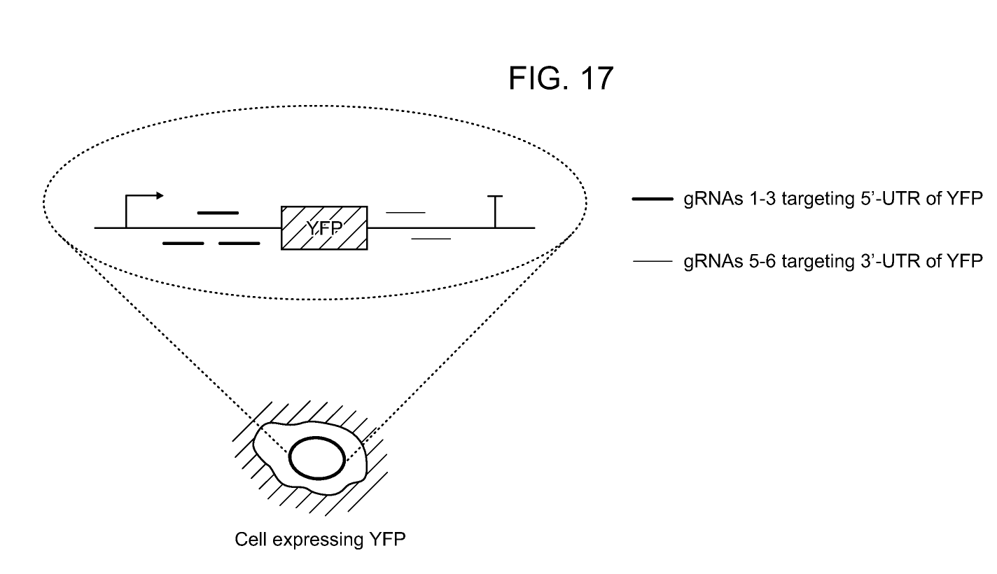

FIG. 17 shows an example of a system in which guide RNAs (gRNAs) cleave and remove a genomically-integrated circuit. In FIG. 17, the gRNAs target the 5′-UTR and 3′-UTR if a YFP reporter that has been stably integrated into cells.

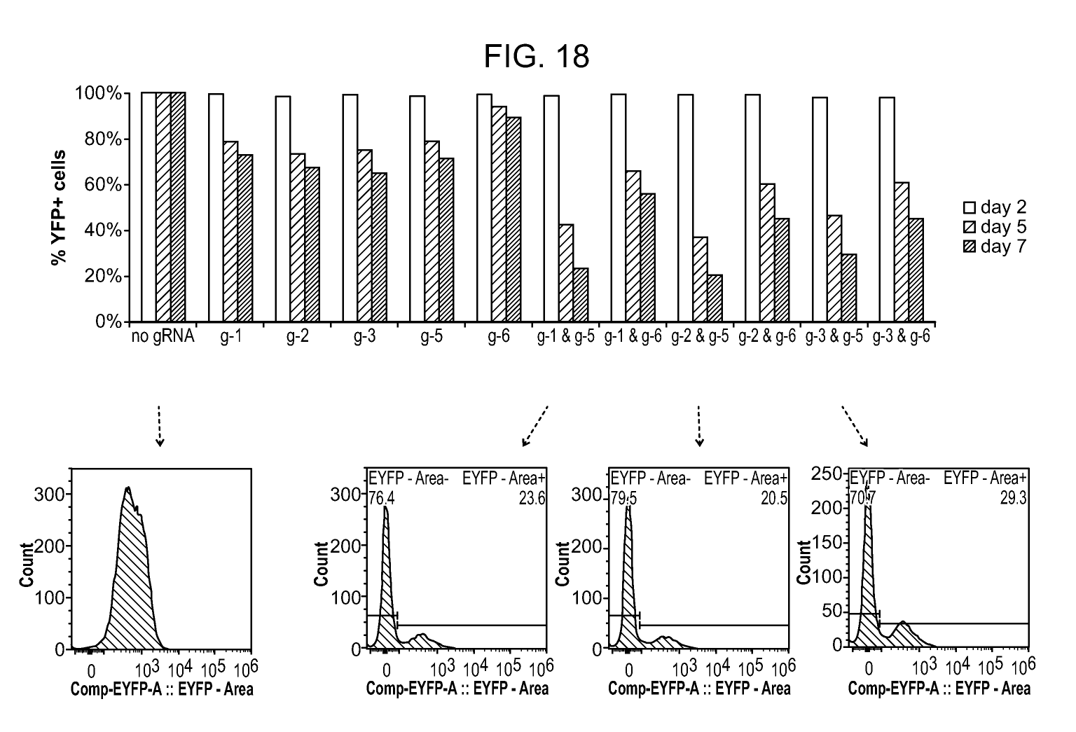

A transient transfection assay was performed to test the removal of a genomically-integrated circuit using CRISPR/Cas9. Vectors encoding a single gRNA and Cas9 were co-transfected along with a reporter plasmid (e.g., expressing BFP) into a cell line that expresses YFP. In some cases, two vectors encoding different gRNAs were transfected along with a reporter plasmid. The cell populations were gated for BFP expression, and the % of YFP+ cells were plotted over time. Different combinations of gRNAs may be used to remove YFP. See FIG. 18.

### REFERENCES

1. Frakes A E, Ferraiuolo L, Haidet-Phillips A M, Schmelzer L, Braun L, Miranda C J, Ladner K J, Bevan A K, Foust K D, Godbout J P, Popovich P G, Guttridge D C, Kaspar B K. Microglia induce motor neuron death via the classical NF-kappaB pathway in amyotrophic lateral sclerosis. Neuron. 2014; 81(5):1009-23. doi: 10.1016/j.neuron.2014.01.013. PubMed PMID: 24607225; PMCID: PMC3978641.
2. Xie Z, Wroblewska L, Prochazka L, Weiss R, Benenson Y. Multi-input RNAi-based logic circuit for identification of specific cancer cells. Science. 2011; 333(6047):1307-11. Epub 2011/09/03. doi: 10.1126/science.1205527. PubMed PMID: 21885784.
3. Saxena P, Heng B C, Bai P, Folcher M, Zulewski H, Fussenegger M. A programmable synthetic lineage-control network that differentiates human IPSCs into glucose-sensitive insulin-secreting beta-like cells. Nature communications. 2016; 7:11247. doi: 10.1038/ncomms11247. PubMed PMID: 27063289; PMCID: PMC4831023.
4. Rogakou E P, Pilch D R, Orr A H, Ivanova V S, Bonner W M. DNA double-stranded breaks induce histone H2AX phosphorylation on serine 139. J Biol Chem. 1998; 273(10):5858-68. PubMed PMID: 9488723.
5. Barretina J, Caponigro G, Stransky N, Venkatesan K, Margolin A A, Kim S, Wilson C J, Lehar J, Kryukov G V, Sonkin D, Reddy A, Liu M, Murray L, Berger M F, Monahan J E, Morais P, Meltzer J, Korejwa A, Jane-Valbuena J, Mapa F A, Thibault J, Bric-Furlong E, Raman P, Shipway A, Engels I H, Cheng J, Yu G K, Yu J, Aspesi P, Jr., de Silva M, Jagtap K, Jones M D, Wang L, Hatton C, Palescandolo E, Gupta S, Mahan S, Sougnez C, Onofrio R C, Liefeld T, MacConaill L, Winckler W, Reich M, Li N, Mesirov J P, Gabriel S B, Getz G, Ardlie K, Chan V, Myer V E, Weber B L, Porter J, Warmuth M, Finan P, Harris J L, Meyerson M, Golub T R, Morrissey M P, Sellers W R, Schlegel R, Garraway L A. The Cancer Cell Line Encyclopedia enables predictive modelling of anticancer drug sensitivity. Nature. 2012; 483(7391):603-7. doi: 10.1038/nature11003. PubMed PMID: 22460905; PMCID: PMC3320027.
6. Huang W Y, Hsu S D, Huang H Y, Sun Y M, Chou C H, Weng S L, Huang H D. MethHC: a database of DNA methylation and gene expression in human cancer. Nucleic Acids Res. 2015; 43(Database issue):D856-61. doi: 10.1093/nar/gku1151. PubMed PMID: 25398901; PMCID: PMC4383953.
7. Gure A O, Tureci O, Sahin U, Tsang S, Scanlan M J, Jager E, Knuth A, Pfreundschuh M, Old L J, Chen Y T. SSX: a multigene family with several members transcribed in normal testis and human cancer. Int J Cancer. 1997; 72(6):965-71. PubMed PMID: 9378559.
8. Landgraf P, Rusu M, Sheridan R, Sewer A, Iovino N, Aravin A, Pfeffer S, Rice A, Kamphorst A O, Landthaler M, Lin C, Socci N D, Hermida L, Fulci V, Chiaretti S, Foa R, Schliwka J, Fuchs U, Novosel A, Muller R U, Schermer B, Bissels U, Inman J, Phan Q, Chien M, Weir D B, Choksi R, De Vita G, Frezzetti D, Trompeter H I, Hornung V, Teng G, Hartmann G, Palkovits M, Di Lauro R, Wernet P, Macino G, Rogler C E, Nagle J W, Ju J, Papavasiliou F N, Benzing T, Lichter P, Tam W, Brownstein M J, Bosio A, Borkhardt A, Russo J J, Sander C, Zavolan M, Tuschl T. A mammalian microRNA expression atlas based on small RNA library sequencing. Cell. 2007; 129(7):1401-14. doi: 10.1016/j.cell.2007.04.040. PubMed PMID: 17604727; PMCID: PMC2681231.
9. Gitzinger M, Kemmer C, El-Baba M D, Weber W, Fussenegger M. Controlling transgene expression in subcutaneous implants using a skin lotion containing the apple metabolite phloretin. Proc Natl Acad Sci USA. 2009; 106(26):10638-43. doi: 10.1073/pnas.0901501106. PubMed PMID: 19549857; PMCID: PMC2700147.
10. Fux C, Moser S, Schlatter S, Rimann M, Bailey J E, Fussenegger M. Streptogramin- and tetracycline-responsive dual regulated expression of p27Kip1 sense and antisense enables positive and negative growth control of Chinese hamster ovary cells. Nucleic Acids Research. 2001; 29(4):e19. doi: 10.1093/nar/29.4.e19.
11. Fussenegger M, Morris R P, Fux C, Rimann M, von Stockar B, Thompson C J, Bailey J E. Streptogramin-based gene regulation systems for mammalian cells. Nat Biotech. 2000; 18(11):1203-8.
12. Weber W, Fux C, Daoud-El Baba M, Keller B, Weber C C, Kramer B P, Heinzen C, Aubel D, Bailey J E, Fussenegger M. Macrolide-based transgene control in mammalian cells and mice. Nat Biotech. 2002; 20(9):901-7. doi: http://www.nature.com/nbt/journal/v20/n9/suppinfo/nbt731_S1.html.
13. Gitzinger M, Kemmer C, Fluri D A, El-Baba M D, Weber W, Fussenegger M. The food additive vanillic acid controls transgene expression in mammalian cells and mice. Nucleic Acids Res. 2012; 40(5):e37. doi: 10.1093/nar/gkr1251. PubMed PMID: 22187155; PMCID: PMC3300003.
14. Hirrlinger J, Requardt R P, Winkler U, Wilhelm F, Schulze C, Hirrlinger P G. Split-CreERT2: temporal control of DNA recombination mediated by split-Cre protein fragment complementation. PLoS One. 2009; 4(12):e8354. doi: 10.1371/journal.pone.0008354. PubMed PMID: 20016782; PMCID: PMC2791205.
15. Feil R, Wagner J, Metzger D, Chambon P. Regulation of Cre recombinase activity by mutated estrogen receptor ligand-binding domains. Biochem Biophys Res Commun. 1997; 237(3):752-7. doi: 10.1006/bbrc.1997.7124. PubMed PMID: 9299439.
16. Sharma N, Moldt B, Dalsgaard T, Jensen T G, Mikkelsen J G. Regulated gene insertion by steroid-induced PhiC31 integrase. Nucleic Acids Res. 2008; 36(11):e67. doi: 10.1093/nar/gkn298. PubMed PMID: 18499713; PMCID: PMC2441784.
17. Hunter N L, Awatramani R B, Farley F W, Dymecki S M. Ligand-activated Flpe for temporally regulated gene modifications. Genesis. 2005; 41(3):99-109. doi: 10.1002/gene.20101. PubMed PMID: 15729687.
18. Jullien N, Sampieri F, Enjalbert A, Herman J P. Regulation of Cre recombinase by ligand-induced complementation of inactive fragments. Nucleic Acids Res. 2003; 31(21):e131. PubMed PMID: 14576331; PMCID: PMC275488.
19. Yang L, Nielsen A A, Fernandez-Rodriguez J, McClune C J, Laub M T, Lu T K, Voigt C A. Permanent genetic memory with >1-byte capacity. Nat Methods. 2014. doi: 10.1038/nmeth.3147. PubMed PMID: 25344638.
20. Canver M C, Bauer D E, Dass A, Yien Y Y, Chung J, Masuda T, Maeda T, Paw B H, Orkin S H. Characterization of genomic deletion efficiency mediated by clustered regularly interspaced palindromic repeats (CRISPR)/Cas9 nuclease system in mammalian cells. J Biol Chem. 2014; 289(31):21312-24. doi: 10.1074/jbc.M114.564625. PubMed PMID: 24907273; PMCID: PMC4118095.
21. Negroni L, Samson M, Guigonis J-M, Rossi B, Pierrefite-Carle V, Baudoin C. Treatment of colon cancer cells using the cytosine deaminase/5-fluorocytosine suicide system induces apoptosis, modulation of the proteome, and Hsp90β phosphorylation. Molecular Cancer Therapeutics. 2007; 6(10):2747-56. doi: 10.1158/1535-7163.mct-07-0040.
22. Kievit E, Bershad E, Ng E, Sethna P, Dev I, Lawrence T S, Rehemtulla A. Superiority of yeast over bacterial cytosine deaminase for enzyme/prodrug gene therapy in colon cancer xenografts. Cancer Res. 1999; 59(7):1417-21. PubMed PMID: 10197605.
23. Beck C, Cayeux S, Lupton S D, Dorken B, Blankenstein T. The thymidine kinase/ganciclovir-mediated “suicide” effect is variable in different tumor cells. Human gene therapy. 1995; 6(12):1525-30. doi: 10.1089/hum.1995.6.12-1525. PubMed PMID: 8664377.
24. Deans T L, Cantor C R, Collins J J. A tunable genetic switch based on RNAi and repressor proteins for regulating gene expression in mammalian cells. Cell. 2007; 130(2):363-72. Epub 2007/07/31. doi: S0092-8674(07)00684-8 [pii] 10.1016/j.cell.2007.05.045. PubMed PMID: 17662949.
25. Wroblewska L, Kitada T, Endo K, Siciliano V, Stillo B, Saito H, Weiss R. Mammalian synthetic circuits with RNA binding proteins for RNA-only delivery. Nat Biotechnol. 2015; 33(8):839-41. doi: 10.1038/nbt.3301. PubMed PMID: 26237515; PMCID: PMC4532950.
26. Muller P Y, Milton M N. The determination and interpretation of the therapeutic index in drug development. Nat Rev Drug Discov. 2012; 11(10):751-61. doi: 10.1038/nrd3801. PubMed PMID: 22935759.
27. Capelli C, Pedrini O, Valgardsdottir R, Da Roit F, Golay J, Introna M. Clinical grade expansion of MSCs. Immunol Lett. 2015; 168(2):222-7. doi: 10.1016/j.imlet.2015.06.006. PubMed PMID: 26092523.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An engineered genetic construct comprising a cassette that comprises (a) a nucleotide sequence encoding a first product of interest, and (b) a nucleotide sequence encoding a second product of interest and a counterselectable marker, wherein expression or activity of the first product of interest is activatable, and wherein the first product of interest modulates excision or degradation of the cassette.

2. The engineered genetic construct of claim 1, wherein the first product of interest is a recombinase.

3. The engineered genetic construct of claim 2, wherein the recombinase is a ligand-dependent chimeric recombinase.

4. The engineered genetic construct of claim 3, wherein the ligand-dependent chimeric recombinase is linked to a mutated human estrogen receptor (ER) ligand binding domain.

5. The engineered genetic construct of claim 3, wherein the recombinase is a split-recombinase that includes a first fragment and a second fragment that when combined form the recombinase.

6. The engineered cell of claim 5, wherein dimerization of the first fragment and the second fragment is inducible.

7. The engineered cell of claim 5, wherein the first fragment is linked to a FKBP domain and the second fragment is linked to FRB domain.

8. The engineered genetic construct of claim 2, wherein the recombinase is selected from tyrosine recombinases and tyrosine integrases.

9. The engineered genetic construct of claim 8, wherein the recombinase is a tyrosine recombinase selected from Cre, Dre, Flp, KD, B2, B3, λ, HK022 and HP1 recombinases.

10. The engineered genetic construct of any one of claim 2, wherein the recombinase(s) is selected from serine recombinases and serine integrases.

11. The engineered genetic construct of claim 10, wherein the recombinase is selected from γ6, ParA, Tn3, Gin, ΦC31, Bxb1 and R4 recombinases.

12. The engineered genetic construct of any one of claims 2-11, wherein the cassette is flanked by cognate recombinase recognition sites.

13. The engineered genetic construct of claim 1, wherein the first product of interest is a nuclease.

14. The engineered genetic construct of claim 13, wherein the nuclease is selected from meganucleases, RNA-guided nucleases, zinc-finger nucleases, and transcription activator-like effector nucleases.

15. The engineered genetic construct of claim 14, wherein the nuclease is a meganuclease selected from intron endonucleases and intein endonucleases.

16. The engineered genetic construct of claim 14, wherein the nuclease is a RNA-guided nuclease selected from Cas9 nucleases and Cpf1 nucleases.

17. The engineered genetic construct of claim 16, wherein the cassette further comprises nucleotide sequences encoding guide RNAs (gRNAs) complementary to the nuclease recognitions sites.

18. The engineered genetic construct of any one of claims 13-17, wherein the cassette comprises cognate nuclease recognition sites.

19. The engineered genetic construct of any one of claims 1-18, wherein the nucleotide sequence of (a) is operably linked to an inducible promoter.

20. The engineered genetic construct of any one of claims 1-19, wherein the nucleotide sequence of (b) is operably lined to a constitutive promoter.

21. The engineered genetic construct of any one of claims 1-18, wherein the nucleotide sequenced of (a) and (b) are operably linked to a single constitutive promoter.

22. The engineered genetic construct of any one of claims 1-21, wherein the second product of interest is a therapeutic molecule or a prophylactic molecule.

23. The engineered genetic construct of any one of claims 1-22, wherein the product of interest is a protein, peptide or nucleic acid.

24. The engineered genetic construct of claim 23, wherein the product of interest is a nucleic acid selected from RNA, DNA or a combination of RNA and DNA.

25. The engineered genetic construct of claim 24, wherein product of interest is a RNA selected from short-hairpin RNAs, short interfering RNAs and micro RNAs.

26. The engineered genetic construct of any one of claims 1-25, wherein the counterselectable marker is a prodrug.

27. The engineered genetic construct of any one of claims 1-25, wherein the counterselectable marker is selected from cytosine deaminases and thymidine kinases

28. A vector comprising the engineered genetic construct of any one of claims 1-27, optionally wherein the vector is a plasmid or a viral vector.

29. A cell comprising the engineered genetic construct of any one of claims 1-27 or the vector of claim 28.

30. The cell of claim 29, wherein the cell is a stem cell or an immune cell.

31. The cell of claim 30, wherein the cell is a stem cell selected from mesenchymal stem cells, hematopoietic stem cells, embryonic stem cells, and pluripotent stem cells.

32. The cell of claim 30, wherein the cell is an immune cell selected from natural killer (NK) cells, NKT cells, mast cells, eosinophils, basophils, macrophages, neutrophils, dendritic cells, T cells and B cells.

33. The cell of claim 32, wherein the cell is a T cell selected from CD8+ T cells, CD4+ T cells, gamma-delta T cells, and T regulatory cells.

34. The cell of claim 32 or 33, wherein the T cell is a chimeric antigen receptor (CAR) T cell or an engineered T cell receptor (TCR) cell.

35. A composition comprising the engineered genetic construct of any one of claims 1-27, the vector of claim 28, or the cell of any one of claims 29-34.

36. A kit comprising the engineered genetic construct of any one of claims 1-27 or the vector of claim 28 and at least one inducer agent and/or counterselective agent.

37. A method comprising introducing into a population of cells the engineered genetic construct of any one of claims 1-27 or the vector of claim 28.

38. A method of delivering to a subject the cell of any one of claims 29-34.

39. An engineered genetic construct comprising a cassette that comprises:
(a) an inducible promoter operably linked to a nucleotide sequence encoding a recombinase; and
(b) a promoter operably linked to a nucleotide sequence encoding a product of interest and a counterselectable marker,
wherein the cassette is flanked by cognate recombinase recognition sites.

40. The engineered genetic construct of claim 39, wherein (a) is upstream from (b).

41. The engineered genetic construct of claim 39 or 40, wherein a terminator sequence is located between (a) and (b).

42. The engineered genetic construct of any one of claims 39-41, wherein (a) comprises at least two inducible promoters, each linked to a different recombinase, and wherein the cassette is flanked by recombinase recognition sites cognate to the different recombinases.

43. The engineered genetic construct of any one of claims 39-41, wherein the nucleotide sequence of (a) encodes at least two different recombinases, and wherein the cassette is flanked by recombinase recognition sites cognate to the different recombinases.

44. The engineered genetic construct of any one of claims 39-43, wherein the nucleotide sequence of (b) encodes at least two counterselectable markers.

45. The engineered genetic construct of any one of claims 39-44, wherein the recombinase(s) is selected from tyrosine recombinases and tyrosine integrases.

46. The engineered genetic construct of claim 45, wherein the recombinase(s) is selected from Cre, Dre, Flp, KD, B2, B3, λ, HK022 and HP1 recombinases.

47. The engineered genetic construct of any one of claims 39-46, wherein the recombinase(s) is selected from serine recombinases or serine integrases.

48. The engineered genetic construct of claim 47, wherein the recombinase(s) is selected from γδ, ParA, Tn3, Gin, ΦC31, Bxb1 and R4 recombinases.

49. The engineered genetic construct of any one of claims 39-48, wherein the product of interest is a therapeutic molecule or a prophylactic molecule.

50. The engineered genetic construct of any one of claims 39-49, wherein the product of interest is a protein, peptide or nucleic acid.

51. The engineered genetic construct of claim 50, wherein the product of interest is a nucleic acid selected from RNA, DNA or a combination of RNA and DNA.

52. The engineered genetic construct of claim 51, wherein product of interest is a RNA selected from short-hairpin RNAs, short interfering RNAs and micro RNAs.

53. The engineered genetic construct of any one of claims 39-52, wherein the counterselectable marker is a prodrug.

54. The engineered genetic construct of any one of claims 39-52, wherein the counterselectable marker is selected from cytosine deaminases and thymidine kinases.

55. A vector comprising the engineered genetic construct of any one of claims 39-54, optionally wherein the vector is a plasmid or a viral vector.

56. A cell comprising the engineered genetic construct of any one of claims 39-54 or the vector of claim 55.

57. The cell of claim 56, wherein the cell is a stem cell or an immune cell.

58. The cell of claim 57, wherein the cell is a stem cell selected from a mesenchymal stems, hematopoietic stem cells, embryonic stem cells, and pluripotent stem cells.

59. The cell of claim 57, wherein the cell is an immune cell selected from natural killer (NK) cells, NKT cells, mast cells, eosinophils, basophils, macrophages, neutrophils, dendritic cells, T cells and B cells.

60. The cell of claim 59, wherein the cell is a T cell selected from CD8+ T cells, CD4+ T cells, gamma-delta T cells, and T regulatory cells.

61. The cell of claim 59 or 60, wherein the T cell is a chimeric antigen receptor (CAR) T cell or an engineered T cell receptor (TCR) cell.

62. A composition comprising the engineered genetic construct of any one of claims 39-54, the vector of claim 55, or the cell of any one of claims 56-61.

63. A kit comprising the engineered genetic construct of any one of claims 39-54 or the vector of claim 55 and at least one inducer agent that modulates activity of the inducible promoter(s) of (a).

64. A method comprising introducing into a population of cells the engineered genetic construct of any one of claims 39-55 or the vector of claim 56, wherein the product of interest aids in differentiation, expansion or phenotypic maintenance (persistence) of the cells.

65. The method of claim 64 further comprising culturing cells of the population and producing the product of interest.

66. The method of claim 65 further comprising culturing cells of the population in the presence of an inducer agent, activating the promoter of (a), expressing the recombinase(s) and excising the cassette from the engineered genetic construct.

67. The method of claim 66 further comprising culturing cells of the population in the presence of a counterselective agent and killing cells that express the counterselectable marker.

68. The method of claim 67, wherein less than 10% of the cells of the population comprise the cassette following the step of culturing cells of the population in the presence of a counterselective agent.

69. The method of claim 67 further comprising delivering cells of the population to a subject.

70. A method comprising introducing into a population of cells the engineered genetic construct of any one of claims 36-55 or the vector of claim 56, wherein the product of interest aids is a therapeutic molecule and/or a prophylactic molecule.

71. The method of claim 70 further comprising delivering cells of the population to a subject.

72. The method of claim 71 further comprising exposing the subject to an inducer agent, activating the promoter of (a), expressing the recombinase(s) and excising the cassette from the engineered genetic construct.

73. The method of claim 72 further comprising exposing the subject to a counterselective agent and killing cells that express the counterselectable marker.

74. The method of claim 73, wherein less than 10% of the cells of the population comprise the cassette following the step of exposing the subject to a counterselective agent.

75. A method comprising delivering to a subject the cell of any one of claims 56-61.

76. An engineered genetic construct comprising a cassette that comprises:
(a) an inducible promoter operably linked to a nucleotide sequence encoding a nuclease; and
(b) a promoter operably linked to a nucleotide sequence encoding a product of interest and a counterselectable marker,
wherein the cassette comprises cognate nuclease recognition sites, optionally flanking the cassette.

77. The engineered genetic construct of claim 76, wherein (a) is upstream from (b).

78. The engineered genetic construct of claim 76 or 77, wherein a terminator sequence is located between (a) and (b).

79. The engineered genetic construct of any one of claims 76-78, wherein (a) comprises at least two inducible promoters, each linked to a different nuclease, and wherein the cassette comprises nuclease recognition sites cognate to the different nucleases.

80. The engineered genetic construct of any one of claims 76-79, wherein the nucleotide sequence of (a) encodes at least two different nucleases, and wherein the cassette comprises nuclease recognition sites cognate to the different nucleases.

81. The engineered genetic construct of any one of claims 76-80, wherein the nucleotide sequence of (b) encodes at least two counterselectable markers.

82. The engineered genetic construct of any one of claims 76-81, wherein the nuclease(s) is selected from meganucleases, RNA-guided nucleases, zinc-finger nucleases, and transcription activator-like effector nucleases.

83. The engineered genetic construct of claim 82, wherein the nuclease(s) is a meganuclease selected from intron endonucleases and intein endonucleases.

84. The engineered genetic construct of claim 82, wherein the nuclease(s) is a RNA-guided nuclease selected from Cas9 nucleases and Cpf1 nucleases.

85. The engineered genetic construct of claim 84, wherein the cassette further comprises nucleotide sequences encoding guide RNAs (gRNAs) complementary to the nuclease recognitions sites.

86. The engineered genetic construct of any one of claims 76-85, wherein the product of interest is a therapeutic molecule or a prophylactic molecule.

87. The engineered genetic construct of any one of claims 76-86, wherein the product of interest is a protein, peptide or nucleic acid.

88. The engineered genetic construct of claim 87, wherein the product of interest is a nucleic acid selected from RNA, DNA or a combination of RNA and DNA.

89. The engineered genetic construct of claim 88, wherein product of interest is a RNA selected from short-hairpin RNAs, short interfering RNAs and micro RNAs.

90. The engineered genetic construct of any one of claims 76-89, wherein the counterselectable marker is a prodrug.

91. The engineered genetic construct of any one of claims 76-89, wherein the counterselectable marker is selected from cytosine deaminases and thymidine kinases.

92. A vector comprising the engineered genetic construct of any one of claims 76-91, optionally wherein the vector is a plasmid or a viral vector.

93. A cell comprising the engineered genetic construct of any one of claims 76-91 or the vector of claim 92.

94. The cell of claim 93, wherein the cell is a stem cell or an immune cell.

95. The cell of claim 94, wherein the cell is a stem cell selected from mesenchymal stem cells, hematopoietic stem cells, embryonic stem cells, and pluripotent stem cells.

96. The cell of claim 94, wherein the cell is an immune cell selected from natural killer (NK) cells, NKT cells, mast cells, eosinophils, basophils, macrophages, neutrophils, dendritic cells, T cells and B cells.

97. The cell of claim 96, wherein the cell is a T cell selected from CD8+ T cells, CD4+ T cells, gamma-delta T cells, and T regulatory cells.

98. The cell of claim 96 or 97, wherein the T cell is a chimeric antigen receptor (CAR) T cell or an engineered T cell receptor (TCR) cell.

99. A composition comprising the engineered genetic construct of any one of claims 76-91, the vector of claim 92, or the cell of any one of claims 93-98.

100. A kit comprising the engineered genetic construct of any one of claims 76-91 or the vector of claim 92 and at least one inducer agent that modulates activity of the inducible promoter(s) of (a).

101. A method comprising introducing into a population of cells the engineered genetic construct of any one of claims 76-91 or the vector of claim 92, wherein the product of interest aids in differentiation, expansion or phenotypic maintenance (persistence) of the cells.

102. The method of claim 101 further comprising culturing cells of the population and producing the product of interest.

103. The method of claim 102 further comprising culturing cells of the population in the presence of an inducer agent, activating the promoter of (a), expressing the recombinase(s) and excising the cassette from the engineered genetic construct.

104. The method of claim 103 further comprising culturing cells of the population in the presence of a counterselective agent and killing cells that express the counterselectable marker.

105. The method of claim 104, wherein less than 10% of the cells of the population comprise the cassette following the step of culturing cells of the population in the presence of a counterselective agent.

106. The method of claim 104 further comprising delivering cells of the population to a subject.

107. A method comprising introducing into a population of cells the engineered genetic construct of any one of claims 39-55 or the vector of claim 56, wherein the product of interest aids is a therapeutic molecule and/or a prophylactic molecule.

108. The method of claim 107 further comprising delivering cells of the population to a subject.

109. The method of claim 108 further comprising exposing the subject to an inducer agent, activating the promoter of (a), expressing the recombinase(s) and excising the cassette from the engineered genetic construct.

110. The method of claim 109 further comprising exposing the subject to a counterselective agent and killing cells that express the counterselectable marker.

111. The method of claim 110, wherein less than 10% of the cells of the population comprise the cassette following the step of exposing the subject to a counterselective agent.

112. A method comprising delivering to a subject the cell of any one of claims 93-98.

113. An engineered genetic construct comprising a cassette that comprises:
(a) at least one promoter operably linked to at least one nucleotide sequence encoding at least one ligand-dependent chimeric recombinase; and
(b) at least one promoter operably linked to a nucleotide sequence encoding a product of interest and a counterselectable molecule,
wherein the cassette is flanked by cognate recombinase recognition sites.

114. The engineered genetic construct of claim 113, wherein the ligand-dependent chimeric recombinase is linked to a mutated human estrogen receptor (ER) ligand binding domain.

115. A cell comprising the engineered genetic construct of any one of claims 113 and 114.

116. A method comprising delivering to a subject the cell of claim 115.

117. An engineered genetic construct comprising a cassette that comprises:
(a) a promoter operably linked to a nucleotide sequence encoding a first fragment of a recombinase;
(b) a promoter operably linked to a nucleotide sequence encoding a second fragment of a recombinase, wherein the first fragment and the second fragment when combined form a full-length functional recombinase; and
(c) a promoter operably linked to a nucleotide sequence encoding a product of interest and a counterselectable marker,
wherein the cassette is flanked by cognate recombinase recognition sites.

118. The engineered cell of claim 117, wherein the first fragment is linked to a FKBP domain and the second fragment is linked to FRB domain.

119. A cell comprising the engineered genetic construct of claim 117 or 118.

120. A method comprising delivering to a subject the cell of claim 119.
